(19) United States
(12) Patent Application Publication
(10) Pub. No.: US 2026/0240681 A1
(43) Pub. Date: Aug. 20, 2026

(54) WEARABLE WITH TEMPERATURE CONTROL

(71) Applicant: Cube Recovery Company, Los Angeles, CA (US)

(72) Inventors: Aaron Young, Los Angeles, CA (US); Paul Marquis, Los Angeles, CA (US)

(21) Appl. No.: 19/543,643

(22) Filed: Feb. 18, 2026

Related U.S. Application Data
(60) Provisional application No. 63/759,995, filed on Feb. 18, 2025.

(51) Int. Cl. A61F 7/00; A61F 7/02

(52) U.S. Cl. CPC A61F 7/0085; A61F 2007/0055; A61F 2007/0076; A61F 2007/0228; A61F 2007/0257

(57) ABSTRACT

A wearable device having a body-to-liquid heat exchanger, configured to exchange heat between a user’s body and a liquid, the body-to-liquid heat exchanger comprising a conformal bladder defining a serpentine portion of a liquid-flow path; a first conduit placing an inlet to the liquid-flow path in fluid communication with an outlet of a liquid-to-ambient-air heat exchanger; a second conduit placing an outlet of the liquid-flow path in fluid communication with an inlet of the liquid-to-ambient-air heat exchanger; and the liquid-to-ambient-air heat exchanger coupled to the first conduit and the second conduit.

### CROSS-REFERENCE TO RELATED APPLICATIONS

This patent filing claims the benefit of US Provisional Patent Application 63/759,995, titled WEARABLE WITH TEMPERATURE CONTROL, filed 02/18/2025. The entire content of each afore-listed earlier-filed application is hereby incorporated by reference for all purposes.

### BACKGROUND

### 1. Field

The present disclosure relates generally to thermal control devices and, more specifically, to wearables with temperature control.

### 2.Description of the Related Art

Modulating temperature of parts of the human body can be useful in a variety of contexts, from modulating overall body temperature for comfort or to enhance performance, to modulating temperature of individual limbs, joints, or organs in more targeted approaches for therapeutic use cases, for instance. Such temperature modulation may include the application of heat or the removal of heat.

### SUMMARY

The following is a non-exhaustive listing of some aspects of the present techniques. These and other aspects are described in the following disclosure.

Provided is a wearable device having a body-to-liquid heat exchanger, configured to exchange heat between a user’s body and a liquid, the body-to-liquid heat exchanger comprising a conformal bladder defining a serpentine portion of a liquid-flow path; a first conduit placing an inlet to the liquid-flow path in fluid communication with an outlet of a liquid-to-ambient-air heat exchanger; a second conduit placing an outlet of the liquid-flow path in fluid communication with an inlet of the liquid-to-ambient-air heat exchanger; and the liquid-to-ambient-air heat exchanger coupled to the first conduit and the second conduit.

Some embodiments include a method of making, using, or controlling the above embodiments.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a perspective view of a heating and cooling module, in accordance with some embodiments.

FIG. 2 is a perspective view of an ambient to liquid heat exchanger in accordance with some embodiments of the present techniques.

FIG. 3 is an exploded perspective view of the heat exchanger of FIG. 2.

FIG. 4 is another exploded perspective view of the heat exchanger of FIG. 2.

FIG. 5 is an exploded perspective view of another heat exchanger in accordance with some embodiments of the present techniques.

FIG. 6 is an exploded perspective view of another ambient to liquid heat exchanger in accordance with some embodiments of the present techniques.

FIG. 7 is a perspective view of a more complete assembly of the heat exchanger of FIG. 6.

FIG. 8 is a plan view of the heat exchanger of FIG. 6.

FIG. 9 is an exploded perspective view of the heat exchanger of FIG. 6.

FIG. 10 is an exploded perspective view of a body to liquid heat exchanger.

FIG. 11 is another exploded perspective view of the heat exchanger of FIG. 10.

FIG. 12 illustrates a variety of form factors in which the above-described heat exchangers may be used.

FIG. 13 depicts three views of a localized heat exchanger for heating and cooling in accordance with some embodiments of the present techniques.

FIG. 14 is a plan view of a device to heat or cool a user's ankle in accordance with some embodiments of the present techniques.

FIG. 15 is a side view of a device to heat or cool a user's head and neck in accordance with some embodiments of the present techniques.

FIG. 16 illustrates the device of FIG. 15 integrated with a wearable vest.

FIG. 17 is a back view of a vest with a heat exchanger like those described above to heat or cool a user's body in accordance with some embodiments of the present techniques.

FIG. 18 is a front view of the vest of FIG. 17.

FIG. 19 is an example of a computing device by which the above-described heat exchangers may be controlled in accordance with some embodiments of the present techniques.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the fields of heat transfer and medical device design. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Many existing wearable heating and cooling devices for the human body have various deficiencies. Some such devices often require the user to freeze an ice pack and are limited in their use once the ice pack has melted. The process of forming the ice and limited duration can be inconvenient in many use cases and make such devices undesirable. Other examples include various types of battery-powered heating or cooling wearable devices. Some such systems often use Peltier chips located at discrete points adjacent to the human body to directly transfer heat from the human body into the Peltier device through contact. Such systems, however, often suffer from limited contact between rigid, relatively-small-in-surface-area Peltier devices and the curved, expansive surface of various portions of the human body. The user often experiences a small number of small, discrete points of conductive cooling or heating on their body. Other examples include systems with forced liquid convection cooling between tubes that heat or cool the human body, adjacent to the tubes, and a fan-cooled heat exchanger with a Peltier device. These systems, however, often suffer from limited heat transfer area from the tubes arrayed on the human body, and such tubes can be expensive and fragile, making such devices unsuitable for scenarios in which the device is subject to heavy usage or extensive movement by the user. It should be emphasized, though, that none of the foregoing issues with such devices constitute disclaimer or disavowal of the discussed techniques, as various embodiments may be used in conjunction with some of those approaches.

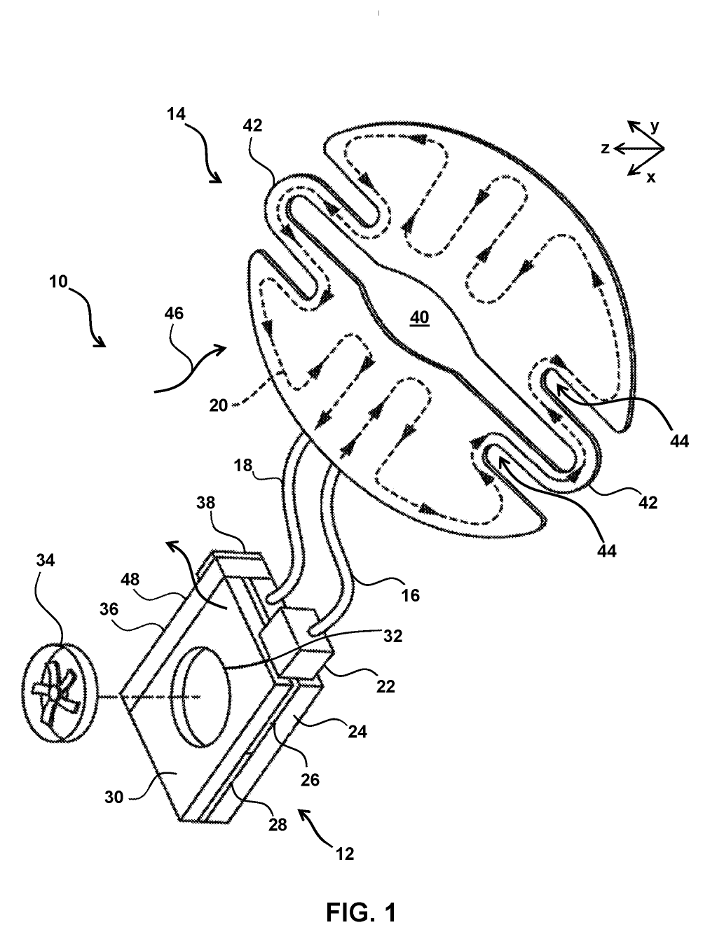

Some embodiments mitigate some, and in some cases all, of the above-described issues with an apparatus depicted in FIG. 1 that, in some embodiments, is a dual heat-exchanger (or quad-heat-exchanger), wearable, personal cooling, and heating device 10 with forced convection cooling or heating. In some environments, the device 10 may include an ambient-to-liquid heat exchanger 12, a body-to-liquid heat exchanger 14, a return conduit 16, and outlet conduit 18. These components may circulate a working fluid, like water, through a serpentine path 20 to remove heat from, or add heat to, a portion of a user's body adjacent the heat exchanger 14. The working fluid may be water, salt water, gallium, water with copper dust, graphene based nanofluids, or the like. In some cases, the working fluid changes phase, and in others, it remains a liquid or a gas.

In some embodiments, the heat exchanger 12 may include a pump 22, a working fluid (e.g., liquid) tank 24, Peltier elements 26 and 28, a heat sink 30 with aperture 32, a battery 36, a controller 38, and various sensors that communicate with the controller 38. In some embodiments, the Peltier elements 26 and 28 may be sandwiched between the heat sink 30 and the tank 24 and all three components may be in thermal communication, for example, with thermal paste, like graphene or graphite based thermal paste, disposed between each of the contact surfaces, above and below the Peltier elements (or in some embodiments, the bottom of the Peltier elements may be in contact with the working liquid).

In some embodiments, the dimensions of the heat exchanger 12 may be relatively compact to facilitate movement of a user when wearing the device 10. In some cases, the heat exchanger 12 may be approximately the size of a deck of playing cards. For instance, with a longest dimension in the X direction less than 5 inches, a thickness in the Z dimension less than 1 inch, and a width in the Y direction of less than 4 inches. In some embodiments, the heat exchanger 12 may have a volume of less than 25 cubic inches, like less than 20 cubic inches, less than 10 cubic inches, or less than 5 cubic inches. The heat exchanger 12 may be positioned on a different part of the body from the heat exchanger 14, which is expected to facilitate expanded range of movement in the areas of the body subject to heating or cooling, as the rigid device 12 will not interfere with motion. Further, it may be desirable to reject heat in a different place from where cooling is applied for device efficiency and comfort.

Various techniques may be used to reduce the dimensions of the device 12, including positioning in aperture 32 to receive the fan 34 by which ambient air is forced down and through fins of the heat sink 30 and against a top surface of a portion of the Peltier elements 26 and 28. Or in some embodiments, the direction of airflow may be reversed. Some embodiments may include additional fans 34 or blowers, e.g., one above each Peltier element 26 and 28. In some embodiments, a battery 36 may be positioned adjacent the above-described stack of components, in some embodiments, on either side with multiple batteries. The battery may be, for example, a lithium-ion battery or other suitable power source. In some embodiments, the controller 38 may be integrally positioned in communication with the fan 34 motor, the Peltier elements 26 and 28, a motor of the pump 22, and a temperature sensor, such as one on the tank 24, the heat sink 30, the Peltier elements 26 and 28, or the heat exchanger 14.

In some embodiments, these components of device 10 may define a closed fluid circuit of a working fluid, such as water or water with glycol. In some embodiments, the working fluid may be driven by the pump 22 through a serpentine path inside tank 24, which may be made from a thermally conductive material, such as aluminum, copper, or the like. That serpentine path in tank 24 may result in the working fluid flowing out of the tank 24 into conduit 18 and from conduit 18 into the serpentine path 20 of heat exchanger 14. When flowing through the serpentine path 20, the working fluid may inflate a portion of the heat exchanger 14 to broaden the path and reduce fluid flow resistance. The working fluid may return out of heat exchanger 14 back through tube 16 to the pump 22 to repeat the process. Depending upon the direction of current flow through Peltier elements 26 and 28, the working fluid may be heated relative to the human body in the tank 24 by pulling thermal energy from the environment or cooled relative to the human body in tank 24 by rejecting thermal energy into the environment through the heat sink 30 with the supportive of forced convection from fan 34 on fins of the heat sink 30.

In some embodiments, the fan 34 may be fully recessed or partially recessed into aperture 32 below a top surface of the heat sink 30 to make the heat exchanger 12 more compact. In some embodiments, the pump 22 may have an impeller that is magnetically coupled to an electric motor that is external to the working fluid flow path, such that the working fluid flow path remains fully closed and not pierced by, for an example, an axle between the motor and the impeller. Or, in some embodiments, there may be an axle with seals suitable for preventing the leakage of the working fluid between the motor and the impeller. Or, some embodiments may use other types of pumps, like a peristaltic pump, to circulate the working fluid. Some embodiments may flow approximately a gallon a minute, or less or more, of the working fluid through the fluid circuit.

Some embodiments may include a relatively flexible heat exchanger 14 that is conformal to the human body and can be inflated by the working fluid partially to define the serpentine path 20. In some embodiments, the heat exchanger 14 is referred to as a bladder. Some heat exchangers 14 may be formed by die cutting two sheets of a flexible, waterproof fabric like polypropylene Pax in the shape illustrated in FIG. 1, aligning those two cut pieces, and thermally welding the two cut pieces together (e.g., melting the polypropylene in discrete areas). In some embodiments, the thermal weld may run along serpentine path 20, on either side of serpentine path 20, for example, defining a channel between one and two centimeters wide with welds on either side of serpentine path 20. In some embodiments, the thermal weld may be between one and five millimeters wide, for example, approximately two or three millimeters wide. Some embodiments may further include a thermally reflective layer such as a metallic foil adjacent a side of the heat exchanger 14 away from the human body to reflect heat away and when cooling or reflect heat back towards the human body when heating. Some embodiments may further include a comfort layer adjacent the human body such as a layer of fabric.

In some embodiments, the heat exchanger 14 may include various features to make the heat exchanger 14 even more conformal to various portions of the human body. The illustrated heat exchanger 14 is expected to be particularly suitable for joints like knees elbows and shoulders. In some embodiments, the aperture 40 may be positioned over a central portion of the joint , like a kneecap or elbow and the flexible bends 42 may be positioned on either side such that the axis of rotation of the joint is parallel to the y-axis as illustrated. Stress relief apertures 44 may reduce crimping that could cut off fluid flow through the serpentine path 20 and accommodate a more expansive range of movement of the user while wearing the heat exchanger 14 against their body. Some embodiments may include anti-crimping structures disposed between the sheets of polypropylene (or other material) constituting the heat exchanger 14, like at bends 42, such as flexible tubes inserted therebetween before welding. Similar arrangements may be used for other portions of the human body , like ankles wrists necks chest back hips and the like.

The heat exchanger 14 may be biased against the wearer's body with a variety of different techniques. Examples include hook-and-loop straps or an ace bandage wrapped around the joint with the bandage holding the heat exchanger 14 against the joint or other portion of the human body. Other examples include a pneumatic or fluidic actuator positioned on a side of the heat exchanger 14 away from the human body such that when another pump drives another fluid or otherwise pressurizes the other fluid in the actuator, the heat exchanger 14 is pressed by the actuator against the human body or grips the human body. Examples of such actuators include soft robotics actuators like those described in a paper titled Modeling of Soft Fiber Reinforced Bending Actuators in IEEE Transactions on Robotics, Volume 31, No. 3, June 2015, the contents of which are hereby incorporated by reference. Other examples include inflatable balloons or sleeves, like those used in automatic blood-pressure cuffs. In some embodiments, one or more such actuators may, for example, run in the y-direction and cause the heat exchanger 14 to wrap around a limb of the wearer when inflated, thereby causing relatively extensive and movement-compliant contact between the heat exchanger 14 and the human body. Other examples include the winch described below.

Some embodiments may use a hard mechanical cage or other apparatus like those described below to hold the heat exchanger 14 in the desired position and protect the heat exchanger 14. Some embodiments may include as an interface between such a shell and the heat exchanger 14 a bladder cage like those depicted in figures discussed below that is resilient but less resilient than the heat exchanger 14, to impart some structure that still accommodates movement of the human body.

Tank 24 may be machined from a solid block of metal, like a solid block of aluminum, or in some cases it may be diecast. Some embodiments may include a diecast tank 24 and a diecast heat sink 30, for example, joined with a hinge running in the x-direction with an axis of rotation parallel to the x-direction, which in some cases may be formed in a single shot. The diecast hinged component may have the Peltier elements 28 and 26 (or other thermoelectric devices) positioned adjacent to tank 24, and then the heat sink 30 may be sandwiched on top, by pivoting about the hinge, thereby providing a relatively low-cost method of manufacturing components of the heat exchanger 12.

In some embodiments, the module device 10 may be used in a vest, knee-brace, ankle, brace, head band, arm-band, or other garments like those discussed below. In some embodiments, there may be one or more such modules, like 2, 4, 6, or more. In some embodiments, different regions of the wearer's torso may be cooled by different heat exchangers 14, which in some cases may be chained in a serial working fluid flow path, or may be in parallel, in some cases with different heat exchangers 12 coupled to different ones of the heat exchangers 14 in parallel flow paths that do not communicate with one another through fluid flow of the working fluid.

In some embodiments, the vest or other configurations described here may include a phase change material, like a wax, or a chilled ice pack, or a thermally reactive consumable, such as those based on cellulose, iron, activated carbon, vermiculite, and salt. Some embodiments may include a heating pouch that augments the operation of the device 10 and may be swapped out as consumables when used. Some embodiments may augment the operation of the device 10 with a compressed inert gas cartridge, like a CO2 (carbon dioxide or other examples described below) cartridge that releases into the aperture 32 to impart a boost of additional cooling, for example, in a hot ambient environment, for instance, upon being triggered by the controller 38 responsive to detecting such an environment, to provide a cooling boost in difficult ambient environments. These cartridges may be replaceable and controlled by the controller 38 through an electrically actuated valve that causes them to release into the aperture 32 when a boost of cooling is needed. Some embodiments may recirculate the formerly compressed gas back across the heat exchanger for multiple cycles, as that gas may be cooler than ambient for multiple such cycles. For instance, some embodiments may include a gas-flow channel (like a manifold or duct) that receives warmed exhaust gas from an edge portion of the heat exchanger (or a top-central portion, depending on the direction of fluid flow) after that gas has passed along and through fins of the heat exchanger 30. In some cases, the gas-flow channel may loop back around to a gas-inlet portion (e.g., the central part above fan 34 if exhausted over the edge or vice versa) and direct gas flow back against the heat exchanger 30 for multiple cycles. Some embodiments may include a thermostat-driven baffle (e.g., actuated by a thermocouple or a motor at the direction of controller 38 responsive to temperature sensor readings of gas circulating) that opens the gas-flow loop, causing ambient air to be ingested and then exhausted, e.g., in response to detecting that the formerly pressurized gas has reached ambient temperature or has warmed to some threshold temperature.

In some embodiments, the vest may include a magnetically coupled interface to receive the heat exchanger 12 with the aperture 32 facing outward away from the human body, adjacent to the exterior portion of the vest, so it can be clipped to the body. Some embodiments may further include an exhaust vent positioned to receive air coming off of the heat sink 30 and vent it out of the garment, for example, in use cases in the firefighting industry to vent hot air away from the user's body. Some embodiments may include a torsion hole in the vest, for example, in the middle of the user's back, to prevent or impede wrinkling or crimping when the user moves their body. Additionally, or alternatively, some embodiments may include a photovoltaic panel, such as a flexible panel mounted on the back or front of a vest, to charge the battery.

In some embodiments, the battery 36 may be inductively or magnetically charged to extend the battery life. For example, some embodiments may include an inductive charging receptacle, like a coiled wire coupled to the controller 38 to deliver power to the battery 36. In some embodiments, this inductive charging receptacle may be positioned, for example, on the user's back, such that when they sit in a seat of, for example, a delivery vehicle, the battery is charged through a complimentary inductive charging pad with another coil positioned on the driver's seat of the vehicle. In this manner, it is expected that such a user may have their battery charged when driving between houses or between tasks, and then draw upon the battery to maintain their comfort when they leave the vehicle, allowing a battery of modest size to last all day through repeated charging between stops. In some vest use cases, embodiments, such as those designed for farmers or others working outdoors, may include panels for photovoltaic charging. Some embodiments may integrate the modules into industrial equipment and clothing such as welding gear, hazmat suits etc.

In some embodiments, the controller 38 may control the Peltier elements 26 and 28 independently to improve their efficiency. In some embodiments, when Peltier elements are run at maximum capacity, they may operate in a less efficient regime. Some embodiments may cycle between activating Peltier elements 26 and 28 with less than 100% duty cycle on each (e.g., each being between 20 and 80% of the time when cooling or heating), and each being out of phase with the other, such that one of the Peltier elements 26 or 28 is recovering closer to ambient temperature, while the other is driving heating or cooling, and vice versa. Some embodiments may include an array of more than 2, like 4, 6, 8, or more Peltier elements positioned adjacent different portions of the working-fluid flow channel in device 12, and some embodiments may selectively engage different subsets of these Peltier elements (e.g., with less than 100% duty cycles and out of phase in a periodic pattern). In some cases, by the duty cycle and driving voltage or current may be modulated by controller 38 to increase cooling or heating applied.

In some embodiments, the control algorithm may produce outputs that control the fan motor 34, the motor of the pump 22, each of the Peltier elements 26 and 28, and a user interface, like on a smartphone or a wearable computing device like a watch. In some embodiments, the controller 38 may receive inputs from an ambient temperature sensor, wearable computing devices (like a watch with a pulse sensor, a gait sensor, a temperature sensor, a blood glucose sensor, a blood oxygen level sensor, a sleep sensor, or the like), a liquid temperature sensor coupled to the tank 24 or immersed in the working liquid, and the like. In some embodiments, control of the various motors may be implemented with pulse width modulation.

Some embodiments may execute a control algorithm that targets a perceived temperature of the user, which may be different for different people in different environments. Some embodiments may actively learn to control the above-described outputs to maintain a perceived temperature that the user desires. Some embodiments may train a machine learning model executed by the controller based upon biometrics sensed, for example, by a smartwatch or other biometric sensors, and adjustments by a given user. For example, some embodiments may learn to minimize the amount of active user adjustment to target a comfortable perceived temperature of the user.

As noted, the module device 10 may have a variety of use cases, including applying heating or cooling to a knee, a wrist, an ankle, a torso, a neck, a quadricep, a hamstring, a calf, foot, a hand, ears, head, or the like. In some embodiments, the device 10 may be suitable for use when the user is ambulatory and engaging in their work. Various verticals are expected to find the above-described device as useful, including in industrial applications, like those servicing airline equipment on a tarmac, those engaged in delivery in hot or cold climates of packages, tactical use cases in which cooling is applied or heating is applied under a ballistic vest, medical use cases in which therapeutic heating and cooling is applied, and virtual reality use cases in which heating or cooling is applied responsive to gameplay, for example, heating or cooling the user when their character moves into a hot or cold environment, respectively, to increase immersiveness of the game. Some embodiments may use the device 10 in motorcycle, construction, military, or bike helmets to cool or heat the user’s head. Some embodiments may enhance workouts by cooling the user’s hands, e.g., through a mitt or glove, or by having heat exchanger 14 wrapped around a handle on a treadmill or other exercise equipment. Other use cases may involve cooling or heating things other than the human body, like heating or cooling a food or beverage product, a pharmaceutical, or a biological substance (like blood samples). Contemplated use cases further include cooling electronic devices, like laptop computers, thermal camera sensors, and the like.

Millions of Americans suffer from chronic injuries every year that may benefit from therapeutic icing. A cryo-pod, an ice bath, and an ice bag may be used to recover from a chronic injury. Some methods of recovering from a chronic injury may include non-portability of a cryo-pod, waste of water in an ice bath, single usage of an ice bag, toxic chemicals in an ice bag, and assistance that may be required with an ice bag. Thus, there is a need for a wearable therapeutic device that is portable, does not waste water, does not use toxic chemicals, is reusable, and does not require an assistant to be physically present (which is not to suggest embodiments are limited to implementations that address all or any of these needs or that any other feature is limiting). Some embodiments mitigate or fully address all of these issues, while others mitigate a subset or other issues. Some embodiments include a device and a method that provide temperature-controlled therapy. Some embodiments include a device and a method that provide therapeutic recovery programs implemented by temperature control.

Wearable devices may be used in various scenarios ranging from athletic recovery to emergency treatment. Such wearable devices may be shaped and constructed differently to serve the athletic community versus the medical community, though a wearable device that is capable of use in multiple situations while being portable and durable is useful as well.

In some embodiments, a wearable therapeutic device includes an exoskeleton structure in which the above module 10 is disposed, the structure comprising a top segment and a bottom segment, the top segment and bottom segment being coupled together at a hinge to move the top segment and bottom segment relative to each other. The wearable therapeutic device may also include an intermediate support sleeve structure encompassing at least a portion of the exoskeleton structure and a rigid outer shell configured to encompass the intermediate support sleeve and the exoskeleton structure, wherein the rigid outer shell includes a winch system.

An exoskeleton composition may be made of a lightweight metal (like an aluminum alloy, titanium alloy, magnesium alloy, etc.), composite materials (like graphene composites, carbon nanotube composites, carbon fiber composites, Kevlar ™ composites, fiberglass composites, etc.), or plastic (e.g., ABS, Nylon, PET, PEEK, etc.) to provide a structural support for the wearable device to provide various therapies. The material used may have a specific strength of between 100 kN m/kg and 63,000 kN m/kg, like between 100 kN m/kg and 2500 100 kN m/kg. In some embodiments, the exoskeleton is composed of a durable material capable of withstanding force allowing a user of the wearable to maintain a desired orientation. For example, the exoskeleton structure may provide enough structural support for a wearer to bear his or her entire body weight on the structure.

The exoskeleton structure may be coupled to an intermediate support sleeve structure to provide a layer between the exoskeleton structure and an outer shell. In some embodiments, the intermediate support sleeve is made of a textile material.

In some embodiments, the rigid (e.g., using a material having a Young’s modulus of 1 GPa or greater) outer shell structure is made of a plastic material (e.g., acrylonitrile butadiene styrene (ABS), high impact polystyrene (HIPS), expanded polystyrene (EPS)) and other suitable casing materials for providing support and protection for the wearer and the components within the wearable device.

In some embodiments, the wearable device includes a one or more winch systems for providing an adjustable fit of the wearable device to the body part of the wearer. The winch system may include a cable, a drum, a spindle, and a gear configured to adjust the tightness of the wearable device to be worn around the body part of the wearer by pulling and winding up or down a cable or other tether coupled to another part of the device that moves relative to the winch system as the winch system is tightened or loosened. In some embodiments, the winch system of the wearable therapeutic device is a manual winch, an electrical winch, a mechanical drum winch, an air winch, or any other suitable pulley system.

In some embodiments, Peltier thermoelectric heat pumps (referred to as “Peltier devices” or “Peltier modules” interchangeably) may be used for cooling body parts of the user. The Peltier device may transport heat using energy provided by electrical current flow. The efficiency of Peltier cooling may depend on the temperature difference across the module. When a large temperature difference is built up across the module, the temperature gradient drives heat flux in the reverse direction as the direction that the heat is intended to be transported, and the coefficient of performance may be decreased. These cases are problematic in some use cases due to the unfavorable scaling relation regarding the resistive heating of the Peltier module itself combined with increased heat backflow as compared to the heat transported per unit current, especially since larger current leads to increased resistive heating of the module itself, which in turn, requires more heat in total to be dissipated. Therefore, it is beneficial in some cases to remove heat from the Peltier module (e.g., from a hot side of the module) to keep the temperature of the thermal transport energy destination low, which is not to suggest that all embodiments must do this or that any other description is limiting. In some embodiments, the wearable device cools the module solely with air cooling by ambient, untreated air. However, due to the low thermal conductivity and volumetric heat capacity of air, the cooling provided tends to be insufficient for transporting large quantities of heat at a high rate in some use cases (which is not to suggest that this approach or any other is disclaimed). Some embodiments enhance transfer of thermal energy away from the side of the Peltier module (e.g., expel thermal energy) where heat is being deposited through dissipating thermal energy to the surrounding environment.

In some embodiments, instead of, or in addition to the Peltier devices, an electrocaloric thin-film heat pump may be used. In some embodiments, an electrocaloric thin-film heat pump may be constructed as a laminated stack in which an electrocaloric layer may be positioned between a first electrode layer and a second electrode layer, and the stack may be supported by one or more substrate layers and encapsulation layers. In some implementations, the electrocaloric layer may include a ferroelectric polymer film or a ferroelectric ceramic thin film, and the film may be deposited or laminated on a flexible polymer substrate or on a thin ceramic substrate. In some cases, the electrode layers may include thin metal films, conductive polymer films, or carbon-based electrode films, and the electrodes may be patterned into one or more independently drivable regions so that subsets of the electrocaloric area may be energized at different times. In some implementations, a dielectric passivation layer may be placed over electrode edges to reduce field concentration, and a moisture barrier layer may be laminated over the stack to reduce humidity ingress. In some cases, one or more heat-spreader layers may be bonded to one or both faces of the stack, such as a thin metal foil, a graphite sheet, or a metallized polymer film, so that temperature across the active area may remain more uniform during cycling.

In some embodiments, the electrocaloric thin-film heat pump may operate by cycling an electric field across the electrocaloric layer while managing heat flow to and from external thermal interfaces. In some implementations, a controller may apply a drive waveform that increases an electric field across the electrocaloric layer for a first interval and decreases the electric field for a second interval, and the electrocaloric layer may undergo a temperature rise during at least part of the first interval and a temperature drop during at least part of the second interval. In some cases, the device may include a thermal switching structure that may change an effective thermal conductance between the electrocaloric stack and a first thermal interface and between the electrocaloric stack and a second thermal interface, such that heat transfer may be encouraged to a heat-rejection side during a portion of the “field-on” interval and heat transfer may be encouraged to a heat-absorption side during a portion of the “field-off” interval. In some implementations, thermal switching may be provided by modulating contact pressure between surfaces using an actuator, by using a switchable interfacial layer whose thermal conductance may change with electrical excitation, by using microstructured contact pads that may alternately engage and disengage, or by using a valve-driven fluid path that may alternately couple the stack to one interface and then another. In some embodiments, multiple electrocaloric layers may be stacked and electrically connected in series or parallel while being thermally arranged in a cascade, and the controller may stagger drive phases across layers to shape heat flow and manage peak current.

In some embodiments, for a wearable heating or cooling use case, the electrocaloric thin-film heat pump may be packaged as a liquid-conditioning module that couples to a circulating working liquid that feeds a bladder. In some implementations, a first face of the electrocaloric stack may be bonded to a liquid-side interface that may include a thin microchannel plate, a stamped manifold plate, or a polymer microfluidic plate that may have a thermally conductive face, and the working liquid may flow through channels adjacent that face. In some cases, a thermally conductive and electrically insulating bond layer may couple the electrocaloric stack to the liquid-side interface while maintaining electrical isolation, and temperature sensors may be placed in the liquid path, in the interface plate, or both. In some implementations, a second face of the electrocaloric stack may be coupled to a heat-rejection interface such as a finned heat sink, a lattice heat sink, or a heat-spreading plate coupled to remote fins, and an air mover may force air across the heat-rejection interface. In some cases, the controller may select cycle frequency, duty cycle, and field amplitude based on measured liquid inlet and outlet temperatures, measured skin-side temperature, and available electrical power, and the controller may coordinate those selections with pump speed and fan speed to maintain a target liquid temperature.

In some embodiments, flexibility may be supported by selecting materials and packaging that allow bending while maintaining electrical insulation and thermal coupling. In some implementations, the electrocaloric layer may be a polymer film on a flexible polymer substrate, and the electrode layers may be formed as thin, compliant traces that may be routed in serpentine patterns to accommodate strain. In some cases, the active region may be segmented into multiple tiles mounted on a flexible circuit, where each tile may remain substantially planar while the inter-tile regions may act as hinge zones that bend. In some implementations, a flexible heat-spreader layer may distribute heat from the tiles to a shared liquid interface, and compliant gap fillers or thermally conductive elastomers may be placed at interfaces to maintain contact under flexion. In some embodiments, the thermal switching structure may be implemented with distributed, low-profile actuators or contact mechanisms that may be arranged in zones so that bending may occur between zones while each zone maintains its own switching sequence, and the controller may adjust timing among zones to reduce transient temperature non-uniformity.

Icing may be used as a therapy for reducing inflammation and pain during exercise. Since the human body is mostly water, cooling is energetically expensive given that water has a high heat capacity. Ice packs depend on the enthalpy of fusion of ice to absorb the thermal energy. For wearable electronic devices to provide similar or equivalent cooling as compared to a passive system like an ice pack, a comparable amount of energy that is absorbed by the enthalpy of fusion of ice may be transported away from the body (in some cases, during a comparable amount of time). During an icing session, around ~300 grams of ice may melt over a time period of less than one hour (like less than 30 minutes), leading to the removal of ~1x10^5 J of energy from the body within 600-1200 seconds. Assuming a reasonable coefficient of performance of 1 for the heat pump (like a Peltier thermoelectric heat pump), ~2x10^5 J may be ejected into the environment to provide a comparable amount of cooling as an ice pack, given that the heat dissipated may include the energy used by the heat pump to transport thermal energy away from the body. The amount of heat to be removed from the system is, in some cases, equivalent to the amount of energy required to vaporize on the order of 100 grams of liquid water, which has a latent heat of vaporization nearly an order of magnitude larger than its latent heat of fusion.

Some embodiments offset the large quantity of energy to be dissipated through the use of vaporization of a substance (which may be carried away from the heat pump by advection or through evaporation at the site of the heat pump or heat sink thermally coupled thereto), which may include any or a combination of the following compounds or mixtures: water, alcohol, propylene glycol, glycerin, or any other harmless substance that has a latent heat of vaporization greater than 15000 J/mol or 300 J/g, such as water, with a latent heat of vaporization of 2257 J/g at ambient pressure at sea level. Some embodiments may perform the vaporization within cooling gas applied to the heat sink 30 above. The aforementioned vaporization process leverages the latent heat of vaporization of the substance and can remove more heat per mass and volume than some other approaches, such as those relying on latent heat of fusion (which is not to suggest that such approaches are disclaimed, as both approaches could be combined in some embodiments). Additionally, air may be transported through the device for the purpose of carrying the vapor from vaporization of the working fluid by the device to the surrounding environment, wherein thermal energy is removed through a combination of (advection driven) convective cooling and evaporative cooling. The advection may be provided by a fan, blower, pump, motor, compressor, or any other system that facilitates bulk transport of gas, air, and/or other fluid, for the purpose of removing the vapor and carrier gas (like ambient air) that has absorbed heat from the device. The energy balance is expected to lead to a greater capability to cool given the limited quantity of energy that is available to power wearable devices, especially due to the lack of need to recondense the harmless substance that is being vaporized and ejected into the surrounding environment external to the thermodynamic system of interest.

In some embodiments, the wearable device includes a heat dissipation system that uses both an advective flux coupled to heat transfer and a phase transition to carry thermal energy away from the heat sink. In some cases, this system may be integrated into the wearable devices described in the documents incorporated by reference. The system may use the vaporization of a harmless substance (like water) to absorb heat at the sites where thermal energy from the body and/or from resistive heating of electronic components are transported to, and uses advection to carry the vaporized substance away.

Some embodiments function with a variety of control systems (examples of which are described in the documents incorporated by reference and elsewhere herein), such as intermittent or alternating power of vapor according to time intervals or measurement of temperature, that is vapor controlled by a thermo-sensor activated or deactivated at specified temperatures; including vapor control of system, such as deactivating the device power when vapor level is empty, and/or generating a display message to “fill or refill” vapor source, for example. Some embodiments may operate at less than 24 volts, for example less than or equal to 12 volts, to mitigate weight, noise, and thermal load from higher-voltage components.

In some cases, the present techniques may be integrated with the devices and processes described in US Patent Applications 18/359,848; 17/520,479; 17/725,229; 63/244,073; and 63/310,046, the contents of which are hereby incorporated by reference.

In some embodiments, a wearable device for providing therapeutic recovery can be coupled with a profile that is set by a person (e.g., a user/wearer of an embodiment, a trainer of the user, etc.) with a recovery program for a user (e.g., an athlete), where the athlete inputs progress (e.g., temperature, time, diet, sleep, stress level, etc.). For example, an athlete has a knee injury, and his trainer wants him to adhere to a recovery program that includes repeated icing and heating of the knee. The athlete may some embodiments that include an integrated communication device that allows the trainer to control/modify/oversee the recovery of the athlete’s knee. In use, the athlete may apply the wearable device to his knee and turn the wearable device on. The trainer may then remotely configure a 20-minute recovery program that includes the wearable device applying heat for 5 minutes, cooling for 5 minutes, and so on until the 20 minutes is up. Alternatively, the athlete may utilize an onboard display to manually configure the wearable device, for example, if the wearable device is too hot or cold for his or her comfort.

In some embodiments, a wearable device for providing therapeutic recovery is wearable on one or more portions of a wearer’s body and provides icing or heating in intervals. For example, a user may program an embodiment to provide heat and/or cold (e.g., icing) in user-definable time intervals (e.g., 15 minutes, 20 minutes). Some embodiments may also be operated manually through a device interface or an application or app (e.g., an application configured to communicate via a Bluetooth ™ (or other forms of communication) of a mobile computing device upon which the application runs with a similar interface on the wearable device).

In some embodiments, the wearable device (or server or mobile computing device with which is communicates) securely transmits a message to another person (e.g., a doctor, a physical therapist, a trainer) and securely receives messages from the same. The message may concern an instruction from the other person to the user of some embodiments, progress of the user, and another type of communication between the other person and the user.

In some embodiments, a wearable device for providing therapeutic recovery may be used on various body parts (e.g., ankle, knee, back, wrist, hip, shoulder, chest, hand, foot, head, abdomen, etc.). Some embodiments may be in the form of a vest. Additionally, the wearable device may be configured for use on multiple body parts (e.g., both a wrist and an ankle), in some cases, both left and right sides or just one.

In some embodiments, a wearable device for providing therapeutic recovery may be used by various people (e.g., a high school athlete, a college athlete, a professional athlete, a person recovering from surgery, a laborer). In some embodiments, a wearable device for providing therapeutic recovery may also be used by a person (e.g., a first responder) on another person (e.g., an injured person). The wearable device may be used for medical purposes (e.g., medical icing) and for enhancing athletic performance (e.g., performance cooling). Medical purposes include providing icing or heating concerning hip replacement surgery, knee replacement surgery, anterior cruciate ligament (ACL) surgery, medial collateral ligament (MCL) surgery, rotator cuff surgery, herniated disc surgery, back pain, wrist pain, hand pain, ankle injury, etc.

In some embodiments, the wearable device for providing therapeutic recovery is portable, powered by a rechargeable battery, provides cold therapy, is wearable, provides heating and/or cooling in user-definable intervals, and is operable via a native application executing on a user’s mobile computing device, like a cell phone.

Expected advantages of some embodiments described herein include the various forms of improvement to thermal efficiency described, temperature control (e.g., adjustability), time control (e.g., adjustability), communication capability (e.g., data updates for efficient assessment), wearability (e.g., long lasting durable mobility), sustainability (e.g., eliminates single-use and reduces water waste), and operability via an application (e.g., allows access via a mobile device). But embodiments are not limited to systems affording these advantages, as various approaches are described that can be used independent and some address other problems, which is not to suggest that any other description is limiting. It is understood that the terms “hotter” and “cooler” refer to temperatures higher and/or lower than a current temperature of the discussed object.

Some embodiments may include a profile in memory that is set by a person (e.g., a user of the invention, a trainer of the user, etc.) with a recovery program for a user of the invention (e.g., an athlete), where the athlete inputs progress (e.g., temperature, time, diet, sleep, stress level, etc.).

Some embodiments may include a wearable that provides icing or heating in intervals. For example, a user of the present invention may program the present invention to provide heat and/or cold (e.g., icing) in user-definable time intervals (e.g., 15 minutes, 20 minutes, etc.). Some embodiments may also be operated manually through a device interface (like buttons or a touchscreen on the wearable device) or an application executing on a client device.

Some embodiments may securely transmit a message to another person (e.g., a doctor, a physical therapist, a trainer, etc.) and securely receives messages from the same. The message may concern an instruction from the other person to the user of the present invention, progress of the user, and another type of communication between the other person and the user.

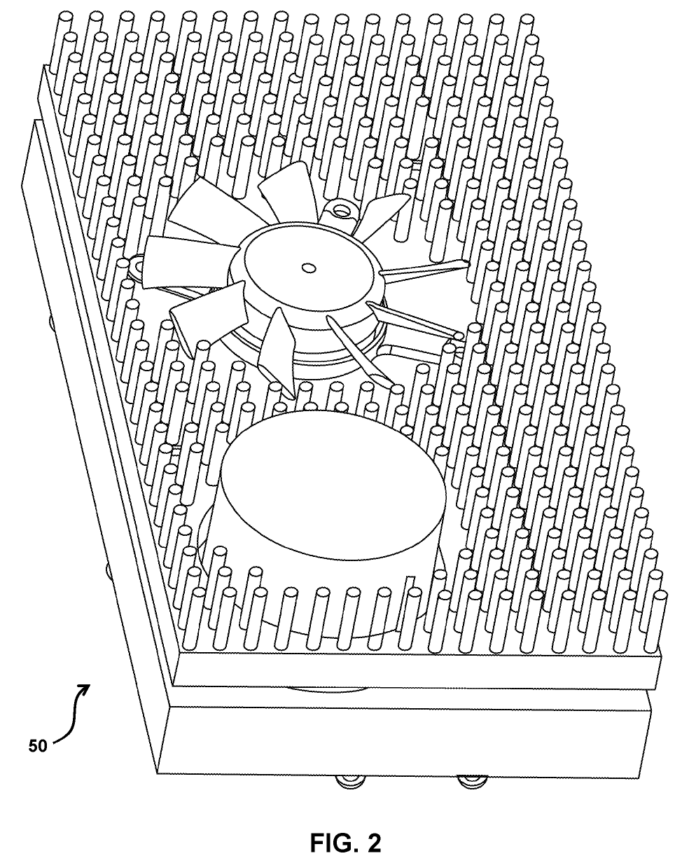

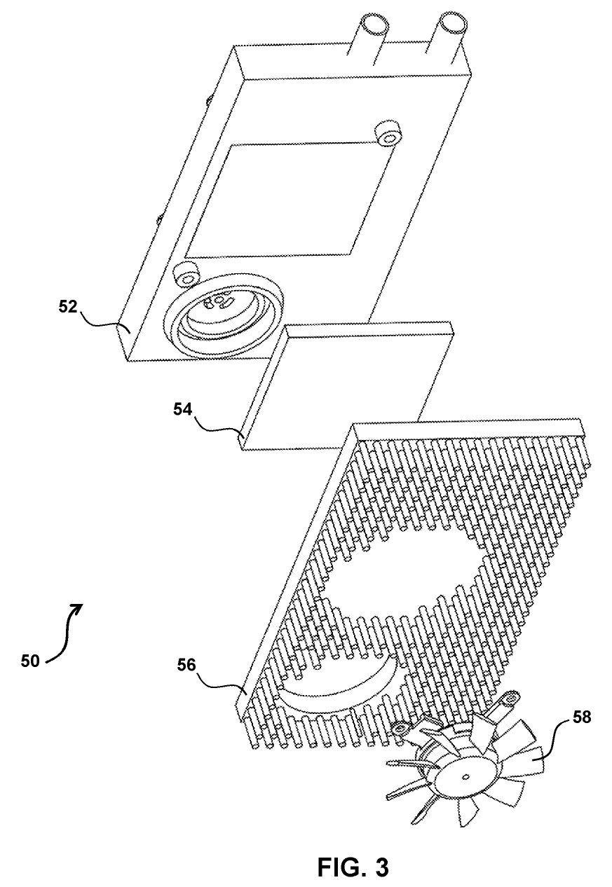

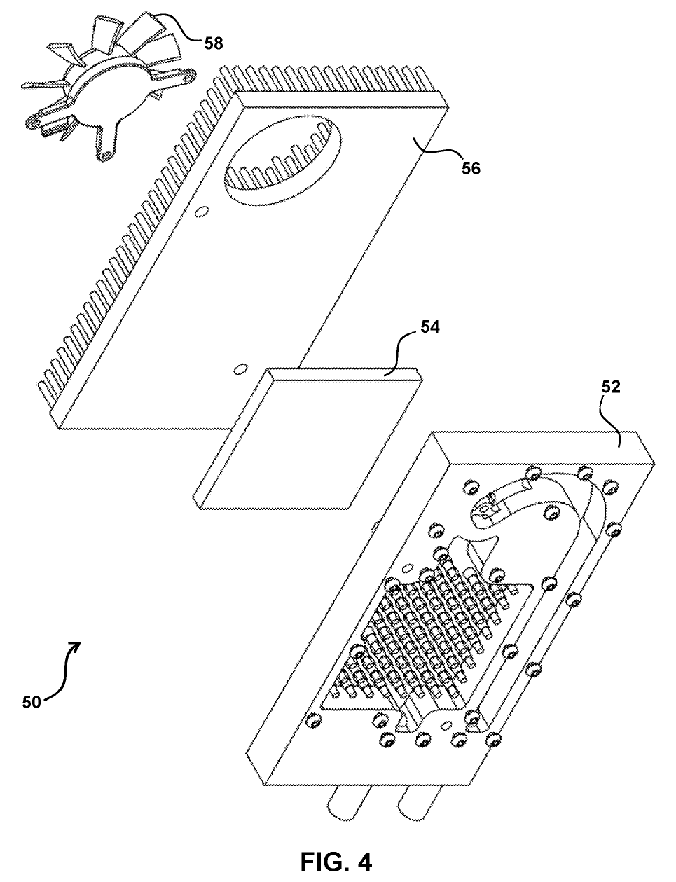

FIGS. 2, 3, and 4 illustrate various views of an ambient to liquid heat exchanger 50. FIG. 2 shows the heat exchanger 50 in assembled form in perspective view, and FIGS. 3 and 4 are two exploded perspective views of the heat exchanger 50. As illustrated, the heat exchanger 50 may serve the role of the heat exchanger 12 described above. In some embodiments, the heat exchanger 50 may include a working fluid tank 52, a Peltier element 54, and a heat sink 56. The tank 52 may be made from, for example, plastic with a metal insert (like a copper or aluminum heat sink similar to heat sink 56, but oriented facing the opposite direction, with fins or protrusions immersed in the working fluid flowing through tank 52, as can be seen in the view of FIG. 4) occupying an aperture in the plastic adjacent the Peltier element 54. That insert may have a similar coating or film to reduce resistance to the flow of heat, for instance, with thermal paste or diamond film. In another example, the tank may be made of magnesium to offer strength with reduced weight, or other materials, like aluminum, titanium, steel, or composites, such as carbon fiber, fiberglass, or the like.

Some embodiments may incorporate materials to reduce thermal resistance at the interface between a Peltier element and a heat sink. Such materials may include thermally conductive pastes, phase change materials, or thin-film coatings that enhance heat transfer by improving surface conformity and reducing interfacial voids.

Thermal pastes may include a matrix material, such as silicone oil, synthetic ester, or polyalphaolefin, which serves as a carrier for thermally conductive fillers. Fillers may include particles of aluminum oxide, boron nitride, zinc oxide, or aluminum nitride, which may exhibit high thermal conductivity while maintaining electrical insulation. In some embodiments, metallic fillers such as silver, copper, or graphene may be incorporated to further increase thermal conductivity. The particle size distribution of the fillers may be controlled to optimize packing density and minimize interstitial voids, thereby enhancing the overall thermal conductivity of the paste. Some thermal pastes may also include phase change materials that soften at operating temperatures, allowing the material to conform to surface irregularities and reduce contact resistance.

In some embodiments, thin-film coatings may be applied to one or both of the interfacing surfaces to enhance heat transfer. One approach may involve chemical vapor deposition (CVD) of a diamond film, which may provide extremely high thermal conductivity. Such a film may be deposited through the dissociation of methane and hydrogen gases in a plasma-enhanced CVD process, leading to the formation of a polycrystalline diamond layer. The thickness of the diamond film may be controlled to balance mechanical stability with thermal performance. Some embodiments may employ diamond-like carbon (DLC) coatings, which may provide improved thermal contact resistance while maintaining lower deposition costs compared to pure diamond films.

Alternative coatings may include metal-based layers such as silver, copper, or gold applied through physical vapor deposition (PVD) or electroplating techniques. These coatings may improve thermal transfer by filling microscopic voids at the interface and reducing the contact resistance between the Peltier element and the heat sink. Some embodiments may incorporate graphene-based coatings, where single or multilayer graphene films are deposited using chemical vapor deposition or solution-based methods. Graphene’s high in-plane thermal conductivity may allow for efficient heat spreading at the interface, reducing localized hot spots.

Some embodiments may include the use of solder-based thermal interface materials, where indium, tin-silver, or gallium-based alloys are applied between the surfaces. These materials may undergo a low-temperature reflow process to form a continuous metallic interface, further reducing thermal resistance. Other embodiments may incorporate carbon nanotube arrays grown directly on one or both surfaces using chemical vapor deposition, which may allow for improved phonon transport across the interface.

A fan 58 with an integrated electric motor may circulate ambient air through conductive paths through metal protrusions (or recesses) arranged (e.g., arrayed) in an array on the heat sink 56. In some cases, the heat sink 56 may be made from copper, aluminum, brass, or other thermally conductive materials. In some cases, the heat sink 56 may include a backplate that is in thermal contact, for instance via thermal paste, direct contact, or chemical-vapor deposition diamond coated films, with a first side of the Peltier element 54, and the working fluid tank 52 may be in thermal contact in a similar manner with a second side of the Peltier element 54, such that the Peltier element 54 removes heat from the tank 52 and liquid flowing therein, and that heat is rejected through the heat sink 56 into ambient air circulated by the fan 58. In some embodiments, the tank 52 may also include an integrated heat exchanger, for instance, implemented with an array of cylindrical protrusions in a fluid path through which a liquid working fluid is circulated. For example, coming in through one of two apertures illustrated in the tank 52 and circulating through the tank and out the other aperture. In some cases, the movement of the working fluid may be driven by a pump shown in figure two as a cylindrical body attached to the heat sink 56 adjacent the fan 58. That cylindrical body may be an electric motor that drives an impeller in a cylindrical recess in the tank 52 to pump the working fluid through the flow path and exchange heat with the working fluid as driven by the Peltier element 54.

Some embodiments may implement a liquid-to-air heat exchanger incorporating three-dimensionally (3D) printed gyroid or similar triply periodic minimal surface (TPMS) structures to enhance heat transfer efficiency. The heat exchanger may consist of a network of interconnected, continuous surfaces forming a highly porous and tortuous geometry, allowing for efficient thermal exchange between a circulating liquid and an airflow. The gyroid structure may be designed with a high surface area-to-volume ratio to maximize contact between the working fluid and the surrounding air while maintaining low pressure drop characteristics.

In some embodiments, the gyroid structure may be fabricated using additive manufacturing techniques such as selective laser melting (SLM), electron beam melting (EBM), or binder jetting when employing metallic materials such as aluminum, copper, or stainless steel. These metals may provide high thermal conductivity while maintaining structural integrity under thermal cycling and fluid pressure variations. In other embodiments, polymer-based materials such as polyamide, polyether ether ketone (PEEK), or resin composites may be employed using stereolithography (SLA) or fused deposition modeling (FDM) processes, with post-processing steps such as metal coating or thermal spraying to improve heat transfer properties.

The liquid-to-air heat exchanger may be configured with multiple fluid channels interwoven within the gyroid structure, where the liquid is directed through the internal pathways while air flows over and through the porous framework. The gyroid topology may enable turbulent flow regimes at relatively low Reynolds numbers, enhancing convective heat transfer by disrupting thermal boundary layers. The pore size and unit cell dimensions of the gyroid structure may be tailored based on the desired flow characteristics, with smaller feature sizes promoting higher heat transfer coefficients while larger structures may reduce hydraulic resistance.

In some embodiments, the gyroid structure may be surface-coated with thermally conductive materials such as diamond-like carbon (DLC), graphene, or a metalized layer using physical vapor deposition (PVD) or electroplating techniques to further improve thermal performance. Alternatively, porous metal foams with gyroid-like geometries may be fabricated via sintering or metal infiltration processes, where molten metal is introduced into a sacrificial polymer template and subsequently solidified to form a highly conductive porous network.

The heat exchanger may be implemented in applications requiring efficient thermal management, such as electronic cooling, battery thermal regulation, or industrial process heat dissipation. Fluid flow paths may be optimized using computational fluid dynamics (CFD) simulations to balance thermal performance with pressure drop considerations. Some embodiments may include variable geometry designs where gyroid unit cell parameters are adjusted locally to accommodate spatial thermal gradients or mechanical constraints. Hybrid implementations may integrate gyroid-based structures with conventional finned heat sinks or microchannel cooling systems to provide multi-scale thermal management solutions.

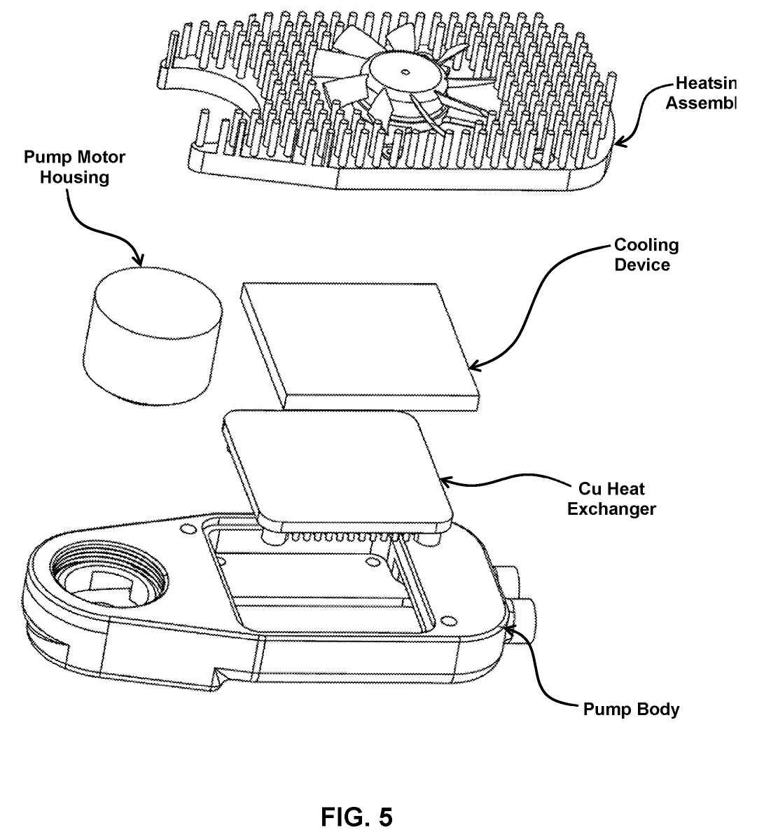

FIG. 5 illustrates another example of a heat exchanging device like the devices 12 and 50 described above with a different physical layout. In some embodiments, the device shown in FIG. 5 may also include a heat-sink assembly, a fan, a cooling device like a Peltier element, a pump motor, a working fluid tank holding the pump body, and a metal heat sink immersed in the working fluid on one side and in thermal contact with the cooling device or heating device, like a Peltier element, on the other. In some embodiments, the opposite side of the Peltier device may be in thermal contact, e.g., via thermal paste or other interfaces described in some embodiments, with a side of the heat-sink assembly As with the devices 12 and 50 described above, the device in FIG. 5 may ingest a working fluid such as water through a first cylindrical aperture shown at the bottom side of the tank, circulate that working fluid with the pump through a path that causes the working fluid to flow around and in contact with an array of cylindrical protrusions in the heat exchanger and out the other illustrated cylindrical aperture at the bottom of the tank labeled pump body in FIG. 5. Heat may flow in a manner like that described above through the Peltier element labeled “cooling device” and the heat sink assembly shown in FIG. 5.

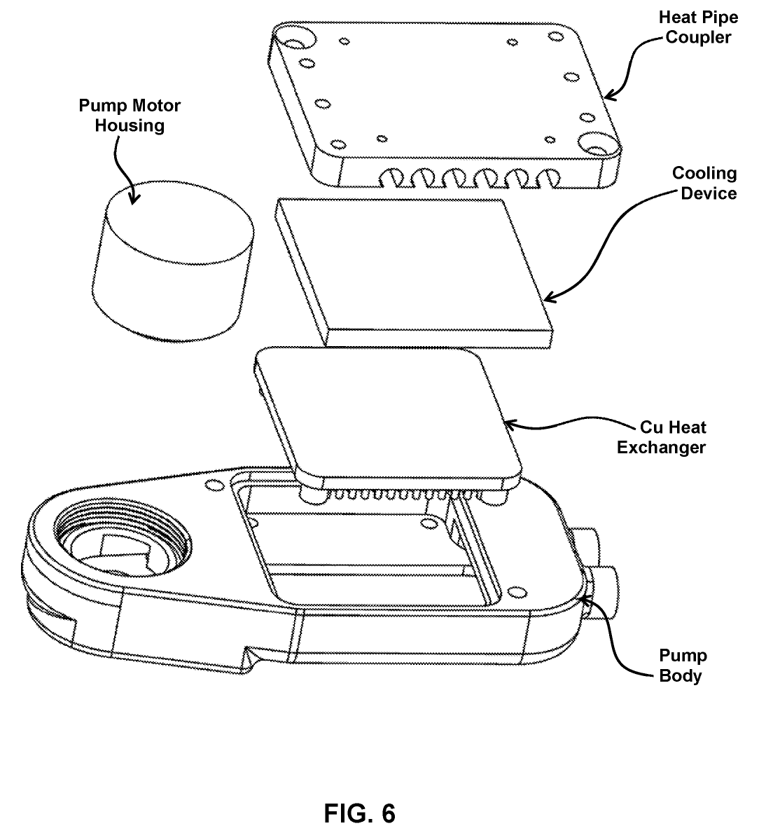

FIG. 6 illustrates another heating and cooling device, like that shown in FIG. 5 and described above with reference to element numbers 50 and 12. Like those other examples, this embodiment may be used in place of the device 12 used as a heat exchanger in FIG. 1. The heat exchanger of FIG. 6 may have components like those described above with reference to FIG. 5, except that in place of the heat sink, a heat pipe coupler may be used. The heat pipe coupler may be made of materials like those described above with reference to the heat sinks. The heat pipe coupler may include an array of generally cylindrical apertures running transversely in a horizontal parallel array adjacent to the cooling device. These cylindrical apertures may be positioned such that a portion of the aperture is exposed to provide direct contact to the cooling device, like a Peltier element, which may also be a heating device depending on the direction of current flow. In some embodiments, heat pipes may be inserted through these apertures to conduct heat away from or toward the Peltier element labeled cooling device. The apertures positioned on the heat pipe coupler may afford direct contact between these heat pipes. In some cases, the heat pipes may have a tubular cylindrical shape like the apertures. In some cases, a portion of this tubular shape may be deformed to provide a flat surface adjacent the cooling device to enhance thermal conductivity. The techniques described above to further enhance thermal conductivity, for instance with various films or thermal paste, may also be applied at this interface. In some cases, the heat pipes may then be connected to and in thermal contact with heat exchangers, for example, at distal ends of the heat pipes, similar to the heat exchangers described above with reference to element numbers 30 and 56. In some cases, multiple heat exchangers may be used. The heat pipes may provide more flexibility in terms of where the weight and volume of the illustrated device is positioned, which may be helpful for positioning the device around various parts of the body, like the knee or ankle or waist or wrist. In some cases, such arrangements may have the effect of shifting the center of gravity of the overall assembly closer to the wearer's body, in some cases positioning that center of gravity within the user's body to provide a more balanced feel as the user moves around.

Some embodiments may include a heat pipe, which may be a passive heat transfer device that employs phase change and capillary action to transport thermal energy efficiently. A heat pipe may include a sealed, evacuated enclosure, an internal wicking structure, and a working fluid that undergoes cyclic phase transitions between liquid and vapor states. The enclosure may be formed from a thermally conductive material such as copper, aluminum, or stainless steel, which facilitates heat transfer between external heat sources and the internal working fluid. The interior surface of the enclosure may be lined with a wick structure designed to transport liquid condensate back to the heat source via capillary action.

The working fluid within the heat pipe may be selected based on its boiling point and compatibility with the operating temperature range of the application. In some embodiments, water may be used for moderate-temperature applications, while ammonia, methanol, or acetone may be used for lower-temperature applications. Higher-temperature applications may employ sodium, potassium, or lithium as the working fluid. The working fluid is introduced into the heat pipe under a vacuum to eliminate non-condensable gases, allowing for phase change at reduced temperatures and enhancing thermal efficiency.

During operation, thermal energy may be introduced at one end of, or the middle of, the heat pipe, e.g., referred to as the evaporator section. Heat input causes the working fluid to absorb energy and transition from liquid to vapor. The generated vapor then moves through the hollow core of the heat pipe toward the cooler end (or ends), known as the condenser section. At the condenser, the vapor releases latent heat to the external environment, often through attachment to a heat sink or finned structure, and condenses back into liquid form. The condensed liquid may be then transported back to the evaporator section through the wick structure, completing the cycle.

The wick structure may be composed of sintered metal, metal mesh, or axially grooved surfaces, with each configuration influencing capillary pumping performance and liquid transport efficiency. Sintered metal wicks may be formed from porous copper or stainless steel, providing fine pore structures that generate high capillary pressure. Mesh wicks may consist of woven or wrapped metal screens that allow for moderate liquid transport capabilities. Grooved wicks may be machined or extruded into the inner surface of the heat pipe enclosure, providing a lower-resistance liquid return path but relying on orientation-dependent performance.

Some embodiments may include variations such as loop heat pipes or vapor chambers. A loop heat pipe may incorporate a secondary reservoir, or compensation chamber, to enhance liquid return capabilities over long distances or against gravity. A vapor chamber may employ a planar heat pipe design where heat spreading occurs across a two-dimensional surface rather than along a single axial path. Various modifications, such as internal bifurcations, hybrid wick structures, or advanced surface coatings, may be implemented to enhance performance for specific applications.

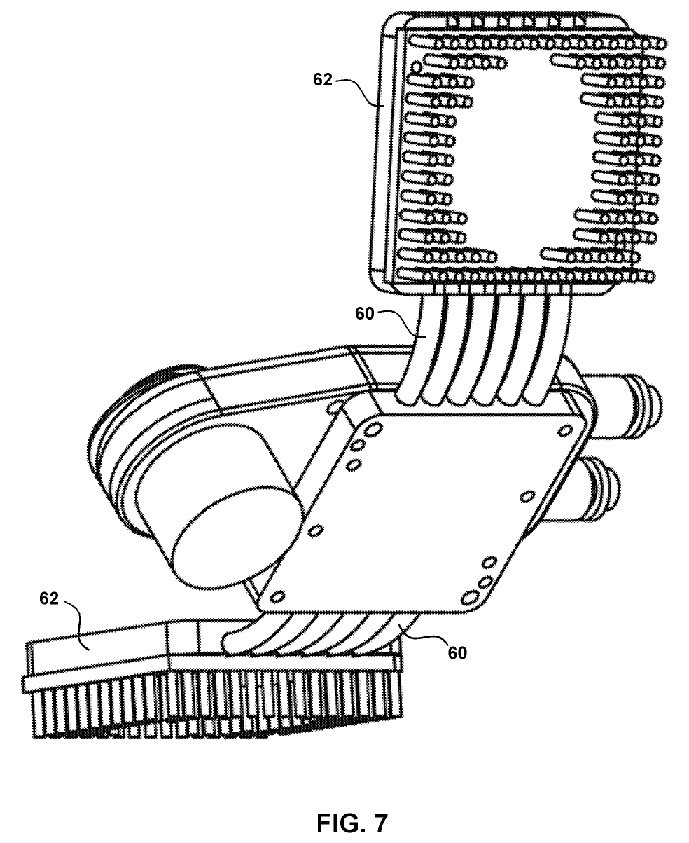

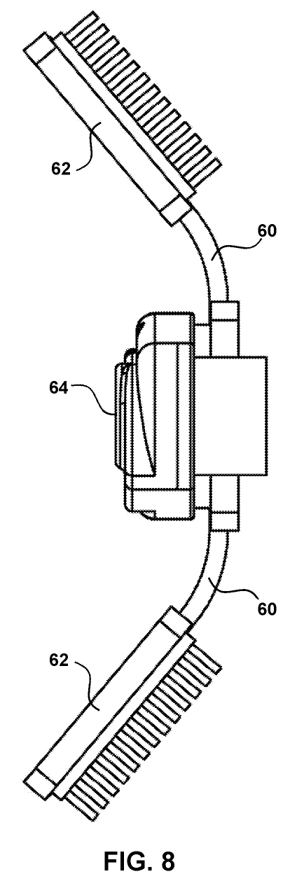

FIG. 7 illustrates an example, along with FIG. 8 of the device of FIG. 6, with an array of heat pipes 60 connected to heat sinks 62 on each side of the device. In some embodiments the heat sinks 62 may have the properties of the heat sinks 30 and 56 described above or those described with reference to FIGS. 5 and 6. In some cases this may include positioning a fan in the illustrated circular gap in the array of cylindrical protrusions of the heat sinks 62. In some embodiments the number of heat pipes 60 may be more than 2, such as between 4 and 8, like 5 or 6. In some cases the diameter of the heat pipe 60 may be between 2 and 10 millimeters, for instance between 3 and 7, or 4 and 6 millimeters. In some cases, the heat pipes 60 may extend along a parallel path, like between 5 and 20 cm. In some embodiments the heat pipes 60 may have a bend with the illustrated curve shown most clearly in FIG. 8. This curve may have the effect of wrapping the heat sinks 62 which may carry a substantial portion of the mass of the overall assembly back around the user's body pushing a center of mass 64 back towards the user's body and in some cases into the user's body relative to assemblies without the bend in the heat pipes 60 or without use of the heat pipes 60. In some cases, the bend may be between 0 and 90 degrees for instance between 10 and 80 like between 15 and 60 or between 45 and 30 degrees. In some cases, the bend may have a radius of between 1 centimeter and 10, like between 2 and 6 centimeters, to avoid crimping the heat pipes 60.

In some embodiments, a heat pipe may include a sealed, thermally conductive enclosure containing a working fluid that undergoes phase transitions to transfer heat efficiently. The enclosure may be evacuated to a low pressure to facilitate phase change of the working fluid at a lower temperature. The heat pipe may include a wick structure along its interior walls, which may be formed from sintered metal, grooves, or a mesh lining to provide capillary action.

In some embodiments, heat is introduced at one end of the heat pipe, referred to as the evaporator section. The applied heat may cause the working fluid within this region to vaporize, forming a low-density vapor that moves toward the cooler end of the heat pipe, referred to as the condenser section. The vapor may flow due to pressure differentials that arise from localized heating and cooling. As the vapor reaches the condenser section, it may release latent heat and condense into liquid form, adhering to the wick structure or the interior surface of the heat pipe.

In some embodiments, capillary action within the wick structure may transport the condensed liquid back to the evaporator section. This capillary-driven return flow may be enhanced by variations in wick design, such as pore size or material composition, to influence fluid flow rate and resistance. Alternative embodiments may employ gravitational forces or external pumping mechanisms to return the liquid to the evaporator section. The cycle of evaporation, vapor transport, condensation, and liquid return may continue indefinitely as long as there is a temperature differential between the evaporator and condenser sections.

Some embodiments may incorporate multiple heat pipes within a heat spreader assembly to enhance thermal management across a surface. The working fluid selection may vary based on the intended operating temperature range, with some embodiments employing water, ammonia, acetone, or specialized refrigerants. Some embodiments may modify the wick structure to optimize heat transport for different orientations, including horizontal, vertical, or inverted configurations. Other embodiments may integrate heat pipes into composite structures, such as vapor chambers, where the heat pipe network distributes heat across a planar surface before dissipating it through attached heat sinks or radiators.

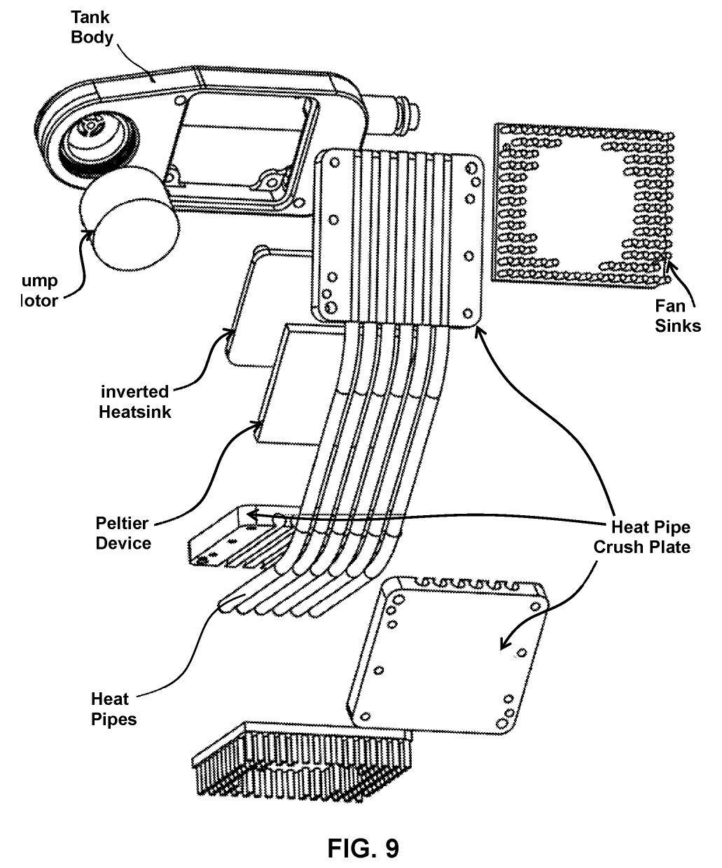

FIG. 9 is an exploded perspective view of the assembly discussed above with reference to FIG. 8. As illustrated, the heat sinks may include heat pipe crush plates, each with the array of generally circular cylindrical holes discussed above that match the outer diameter of the heat pipes, except that the center axis of these holes may be displaced inward from an outer surface of the plates by less than the radius of the heat pipes, for instance, by approximately 98 to 85 percent of that radius. As a result, when the heat pipe crush plates are attached with the heat pipes in place, they may plastically deform sidewalls of the heat pipes, expanding the contact surface from a point to an area and enhancing thermal conductivity. Thus, in some cases, the heat pipe crush plates may laterally compress the heat pipes to deform them. In some cases, the heat pipes may include ribs near where the bend occurs to afford greater flexibility and more extreme bends, for example, like those that are used in a dryer vent pipe.

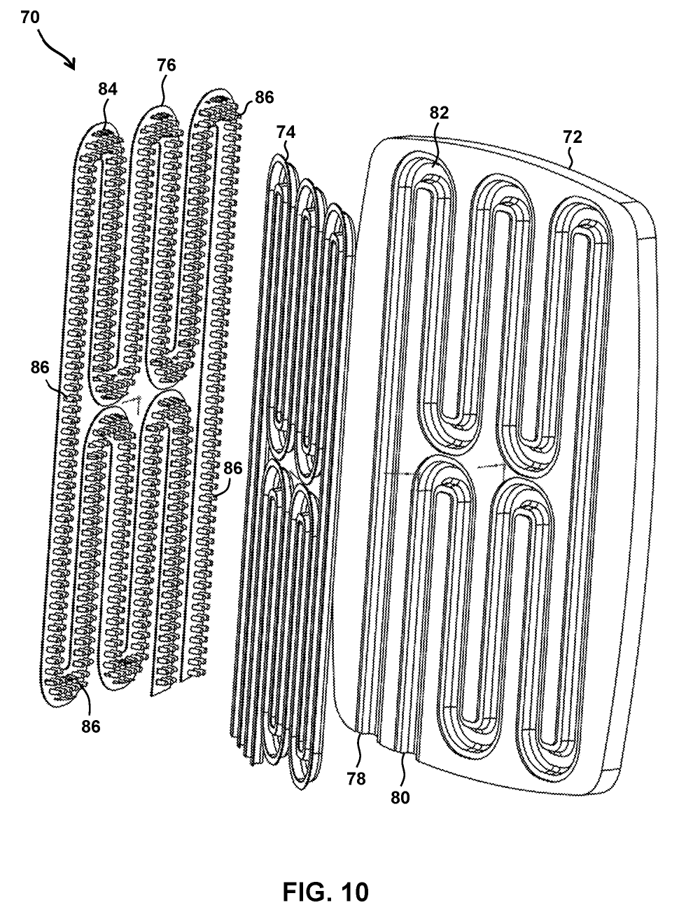

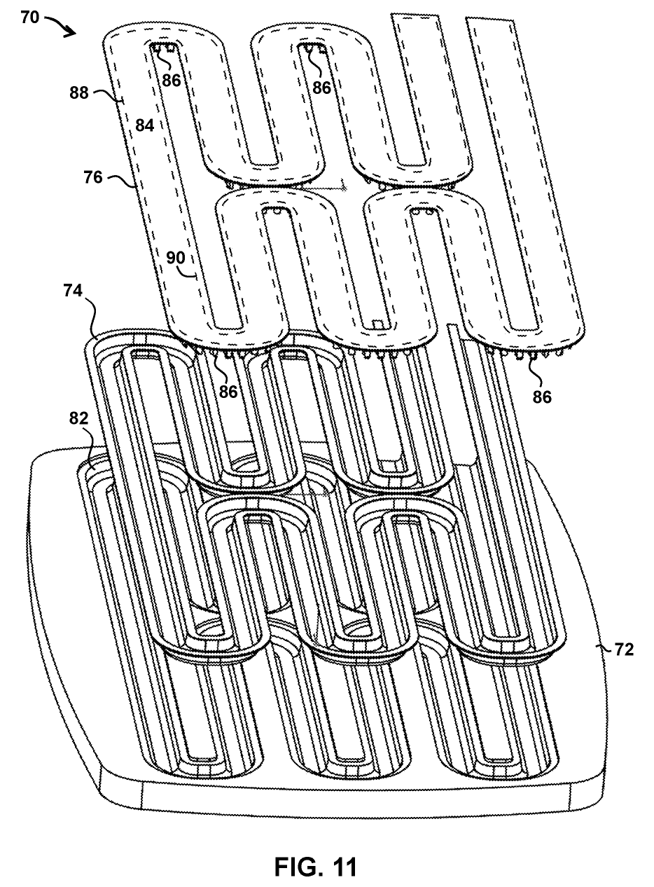

FIG. 10 is an exploded perspective view of another body-to-liquid heat exchanger 70, which may be used in place of the heat exchanger 14 shown in FIG. 1. This heat exchanger may serve to remove heat from the body of a user or add heat to the body of a user. Some embodiments, like the heat exchangers in FIGS. 2-6 described above, may immerse a heat sink in a working fluid to enhance heat transfer. In some cases, this heat sink may be held in a body that insulates other portions to concentrate heat flux with the body, which may also help reduce cost and weight. In some cases, this may add more thermal conductivity in the illustrated channels. The same embodiment is shown from a different perspective, also in an exploded view in FIG. 11. As illustrated, the heat exchanger 70 may include a foam base 72, a spacer tray 74, and a thermally conductive (e.g., made of the materials noted above) heatsink 76 that is flexible. In some embodiments, each of these components may be flexible and conformal to the human body.

In some embodiments, the base 72 may be made of thermally formed foam, like closed cell ethylene-vinyl acetate (EVA) foam, for instance with a channel 82 cut or thermally compression molded into the base 72, with an inlet 78 and an outlet 80 for a fluid flow path that follows the illustrated serpentine path. In some embodiments, a mold assembly may be used, consisting of a lower mold base and an upper mold insert with a raised ridge corresponding to the desired channel geometry. The EVA foam sheet may be positioned between these mold components, and the mold may be heated to a temperature sufficient to soften the material while avoiding excessive degradation. The processing temperature may be selected based on the specific EVA formulation and may typically range between 120°C and 180°C.

As heat is applied, the upper mold insert may be pressed into the softened EVA foam, causing localized compression and densification of the material along the ridge contact area. This compression may result in a permanent deformation, forming a recessed channel with smooth, continuous edges. The depth and width of the channel may be controlled by adjusting the mold geometry, compression force, and processing time.

In some embodiments, vacuum or pressure-assisted molding techniques may be employed to enhance material flow and ensure uniform channel formation. A vacuum may be applied beneath the EVA sheet to draw the material into the mold cavity, promoting consistent shaping and reducing the risk of air entrapment. Alternatively, a pressure plate or roller system may be used to provide uniform compression over a broader surface area while allowing for progressive channel formation.

Post-processing steps may include controlled cooling to stabilize the formed channel structure. The molded EVA foam sheet may be cooled within the mold to prevent unwanted relaxation of the material before removal. In some embodiments, surface treatments such as heat sealing, coating, or adhesive lamination may be applied to reinforce the channel and improve mechanical durability.

Additional features may be integrated into the thermal compression molding process, such as embedding reinforcement layers, applying surface texturing, or incorporating multi-layer EVA foam constructions with varying densities. The resulting molded channel may be used for applications such as flexible fluid conduits, soft robotics, custom orthotics, or lightweight structural components in wearable devices.

Some embodiments may incorporate alternative materials to EVA foam based on desired mechanical properties, thermal stability, flexibility, or durability. These alternatives may include other closed-cell foams, elastomeric materials, or composites that exhibit similar or improved characteristics compared to EVA foam. One alternative may include polyethylene (PE) foam, which may be available in cross-linked or non-cross-linked forms. Cross-linked polyethylene (XLPE) foam may exhibit higher durability, improved thermal resistance, and fine cell structures that provide enhanced cushioning and impact absorption. Non-cross-linked PE foam may offer cost-effective flexibility while maintaining chemical resistance and lightweight properties. Polyurethane (PU) foam may be another alternative, available in both flexible and rigid forms. Flexible PU foam may be used for applications requiring soft, conformable cushioning, while rigid PU foam may provide structural support with higher compressive strength. PU foams may exhibit tunable density and resilience based on formulation adjustments, and some embodiments may incorporate viscoelastic or memory foam variants for specialized applications. Neoprene foam, which may be the form of a closed-cell elastomeric material, may be used as well. Some embodiments may incorporate thermoplastic elastomer (TPE) foams, which may exhibit similar flexibility to EVA while allowing for improved recyclability and heat resistance. Silicone foam may be employed in some cases as well. Thermoplastic polyurethane (TPU) foam may provide an alternative with improved abrasion resistance and elasticity. Some embodiments may incorporate expanded thermoplastic polyurethane (E-TPU), which may exhibit superior energy return and lightweight characteristics. Other polymeric alternatives may include expanded polypropylene (EPP) foam and expanded polystyrene (EPS) foam. EPP foam may offer excellent impact resistance and shape recovery.

In some embodiments, the spacer tray 74 may have the same shape and be more resistant to compressive forces to keep the channel open and reduce pinching that could block fluid flow.

In some embodiments, the heatsink 76 may be made of a flexible, thermally conductive material like an injection molded, or 3D printed, thermoplastic urethane, such as ICE 9 ™ available from TCPoly of 448 Ralph David Abernathy Blvd. Suite 10 Atlanta, GA 30312. Other thermally conductive polymers, such as polyphenylene sulfide (PPS) composites, polyether ether ketone (PEEK) blends, or thermally conductive nylon formulations that incorporate ceramic or metallic fillers. In some embodiments, elastomeric materials such as silicone rubber infused with boron nitride, aluminum oxide, or graphite particles may be employed to provide both flexibility and enhanced heat dissipation properties. Additional alternatives may include liquid crystal polymers (LCP) with thermally conductive additives, which may exhibit high-temperature stability and low thermal expansion characteristics. Some embodiments may utilize carbon-filled thermoplastic elastomers (TPEs) or thermoplastic vulcanizates (TPVs), which may offer a balance of mechanical flexibility and thermal performance. Metal-polymer hybrid materials, where a thermoplastic matrix is reinforced with continuous or discontinuous metallic fibers, may also be suitable. These materials may include copper-, aluminum-, or silver-infused polymer composites that allow for improved heat spreading while maintaining moldability. In some embodiments, graphene- or carbon nanotube-infused polymer materials may be used to achieve enhanced in-plane and through-plane thermal conductivity. Other suitable materials may include phase-change composite polymers, which may incorporate microencapsulated paraffin wax or other latent heat storage materials to improve transient thermal management.

Some embodiments may incorporate synthetic spider silk as a flexible, thermally conductive heat sink material. Synthetic spider silk, which may be produced through recombinant DNA techniques, microbial fermentation, or synthetic polymerization, may exhibit a unique combination of high tensile strength, flexibility, and tunable thermal conductivity. In some embodiments, synthetic spider silk fibers may be coated or impregnated with thermally conductive fillers such as boron nitride, graphene, or carbon nanotubes to increase their effective thermal conductivity. The fibers may be woven, braided, or otherwise structured into a high-surface-area mesh or mat that functions as a flexible heat spreader. The thermal transport properties of synthetic spider silk may be further optimized by aligning polymer chains through mechanical stretching, which may improve phonon transport along the fiber length. In some embodiments, electrospinning or wet spinning techniques may be used to produce ultrafine fibers with controlled morphology, which may allow for enhanced heat dissipation while maintaining mechanical flexibility. Some implementations may include hybrid composites where synthetic spider silk is embedded within a thermally conductive polymer matrix, such as polyether ether ketone (PEEK) or thermoplastic polyurethane (TPU), to create a flexible heat sink material with both high mechanical durability and efficient heat spreading capabilities. In other embodiments, synthetic spider silk may serve as a scaffold for in situ deposition of metallic nanoparticles or conductive coatings, forming a bio-inspired composite material with enhanced thermal performance.

Some embodiments may include thermally conductive open cell foam or other thermally conductive porous media inside conductive bladders or heat exchanger to increase turbulent flow and enhance mixing of a working fluid. Examples include the Duocel® Foam Heat Exchangers from ERG Aerospace of Sparks, Nevada. Such insets can also be made out of a synthetic, flexible, thermally conductive TPU, TPE, or the like, and such inserts can be used for a cold side or a hot side of the system.

In some embodiments, the heatsink 76 may be thermally welded (or glued) to the base 72 with the spacer tray 74 included in the illustrated channels 82. For example, forming a watertight weld along the top surface of the base 72, sealing the edges of the channel 82, and thereby forming a conductive path for fluid through the channel 82. In some embodiments, the heatsink 76 may include a sheet 84 with which this seal is made around its edges, and from this sheet 84 there may be a plurality of protrusions, 86, such as heat conductive fins, cylinders, ridges, channels, or other shapes that increase the surface area exposed to a working fluid passing through the channel 82.

In some embodiments, a wearable heating or cooling device may include a bladder assembly that may define both a working-liquid layer and an airflow-distribution layer, and the airflow-distribution layer may be arranged to deliver air adjacent a wearer-facing surface while the working-liquid layer may remain sealed. In some implementations, the bladder assembly may be formed as a multilayer laminate in which a first film may define a liquid boundary, a second film may define an air boundary, and an intermediate layer may define channel features that may separate an air plenum from a liquid plenum. In some cases, the intermediate layer may include a patterned spacer sheet, a molded lattice, or a welded rib pattern that may create one or more air channels that may extend laterally across a body-contact region. In some embodiments, the wearer-facing surface may include a microperforated cover layer that may have an array of small openings, and the openings may be fluidically coupled to the air channels so that air may exit through the openings and sweep along skin adjacent the bladder. In some implementations, the microperforated cover layer may be a skin-contact textile, a perforated polymer film, or a membrane supported by a spacer fabric, and the membrane may be selected to remain comfortable while providing controlled leakage across the contact area. In some cases, the working-liquid layer may remain separated from the airflow layer by a continuous barrier film, and the barrier film may be thermally coupled to the liquid so that the barrier film may substantially track the temperature of the working liquid along the contact area.

In some embodiments, the airflow may be conditioned to reduce undermining of the heating or cooling effect, and the conditioning may be performed by routing the airflow through a thermal conditioning path that may be thermally coupled to the same working liquid that feeds the bladder. In some implementations, an inlet air stream may first pass through a low-profile air-to-liquid conditioning exchanger that may share a portion of the working-liquid flow path or may be placed in thermal contact with a working-liquid manifold, and the inlet air stream may thereby approach a temperature closer to the bladder surface before the air may be delivered to the wearer-facing microperforations. In some cases, a controller may select an airflow rate and may select a pump flow rate together, and the controller may coordinate those selections so that the delivered airflow may reduce a near-skin boundary layer while the bladder surface may remain within a target temperature range. In some implementations, the controller may estimate a risk of undermining by comparing an air inlet temperature, a bladder temperature, and a skin temperature, and the controller may reduce airflow or increase preconditioning when the comparison indicates that air would otherwise drive heat in an undesired direction. In some embodiments, the airflow path may be arranged as a recirculating loop, and a return duct may capture air near an edge seal and return it to a blower inlet so that a larger fraction of delivered air may remain close to the bladder-conditioned temperature rather than mixing with ambient air. In some cases, the edge seal may be formed by a compliant gasket that may conform to skin and may define a shallow cavity above the microperforated layer, and the gasket may include one or more check valves that may allow pressure equalization while still biasing flow to exit through the microperforations.

In some embodiments, the air delivery may be arranged to increase heat transfer by managing both moisture and micro-scale contact without relying on evaporation as a primary cooling mechanism. In some implementations, the airflow may be directed through the microperforations at a low velocity that may be sufficient to remove humid air trapped near the skin while avoiding strong mixing with ambient air outside the sealed perimeter. In some cases, the microperforations may be sized and spaced so that air may exit as many small jets that may disrupt a stagnant boundary layer without creating localized cold or hot spots, and the jets may be diffused by a spacer fabric layer that may spread airflow laterally along the surface. In some embodiments, a humidity sensor may be positioned in a return duct or within an edge cavity, and a controller may adjust airflow based on the humidity sensor to manage condensation risk near a cooled bladder surface. In some implementations, a dew-point estimate may be generated from measured humidity and temperature values, and the controller may reduce airflow, increase air preconditioning, or adjust bladder temperature when the estimate indicates that condensation may occur. In some cases, a hydrophobic microporous membrane may be placed between the air channels and the wearer-facing surface, and the membrane may allow air passage while limiting liquid transport, and the membrane may be supported by a perforated structural sheet that may resist collapse under strap tension. In some cases, air may be drawn from a return plenum adjacent a body-contact region and routed through a desiccant stage before being reintroduced to a supply plenum. In some implementations, the desiccant stage may include a cartridge that may contain a desiccant material such as silica gel, a molecular sieve, or a hygroscopic salt immobilized in a porous matrix, and the cartridge may be positioned in a duct between a blower outlet and the supply plenum or between the return plenum and a blower inlet. In some cases, air may pass through a porous screen or a honeycomb flow straightener before entering the cartridge, and the cartridge may include a seal to reduce bypass leakage around the desiccant bed.

In some embodiments, the airflow delivery layer may be shaped to preserve flexibility and allow motion, and the layer may include features that may prevent channel collapse during bending. In some implementations, air channels may be segmented into multiple zones separated by flexible hinges, and each zone may include a local plenum fed by a manifold that may include flexible branch passages. In some cases, a manufacturing process may form the branch passages by heat welding or radio-frequency welding of film layers around a patterned tool, and the tool may be selected to create repeatable channel heights. In some embodiments, the airflow may be delivered in pulses rather than continuously, and a controller may command a blower to apply a pulse train with a selected duty cycle that may reduce continuous draft while still renewing near-skin air. In some implementations, the controller may synchronize the pulse train with pump modulation so that periods of higher airflow may coincide with periods when the working liquid temperature near the body-contact area may be closer to a target. In some cases, an inertial sensor may indicate a motion state, and the controller may increase pulse frequency during higher motion states when boundary-layer renewal may be expected to vary more across time.

In some embodiments, the airflow delivery may be implemented as suction rather than positive pressure, and the suction may be applied through a set of distributed micro-openings adjacent the wearer. In some cases, such suction may bias the bladder against the user’s body. In some implementations, a negative-pressure cavity may be formed between a microperforated cover and a barrier film that may separate the cavity from the working liquid, and the negative pressure may draw air across skin and into the cavity for return to a conditioning path. In some cases, suction-based flow may reduce outward leakage at the perimeter, and the perimeter gasket may still allow limited inflow of ambient air through controlled inlet ports that may be positioned away from the highest-heat-transfer region. In some embodiments, inlet ports may be routed through a tortuous path or an acoustic baffle that may reduce noise, and a controller may modulate blower speed based on a pressure sensor that may indicate whether the gasket is sealed adequately.

In some embodiments, the airflow and liquid layers may be arranged so that the airflow may transfer heat to or from the wearer primarily through the bladder temperature set by the liquid loop, and the airflow may primarily serve to reduce thermal resistance at the skin interface. In some implementations, an air channel wall may be formed from a thermally conductive polymer composite, and the wall may be thermally coupled to the working liquid through a thin barrier so that the air may approach the bladder surface temperature as it traverses the channel length. In some cases, the air channels may be routed in a serpentine pattern that may increase residence time along thermally coupled walls, and the serpentine pattern may be laid out to maintain flexibility by using wide-radius turns and compliant hinge regions. In some embodiments, a controller may verify that the airflow is not materially shifting net heat exchange away from a desired mode by estimating a net heat flow based on liquid inlet and outlet temperatures, liquid flow rate, and air temperature change across the air path, and the controller may adjust setpoints based on the estimate while continuing to operate within a wearable power budget.

In some embodiments, a wearable heating or cooling device may include a conformal interface layer between a bladder and a wearer that may reduce microscopic air gaps along irregular skin contours by providing a thin, laterally spreading medium that may remain in place during motion. In some implementations, the interface layer may include a sealed microfilm that may contain a low-volatility, skin-compatible fluid or gel, and the microfilm may be arranged as a network of microcells so that the medium may redistribute locally under strap tension to fill voids while limiting bulk flow. In some cases, the medium may be selected to have a thermal conductivity higher than air and a viscosity that may resist squeeze-out, and the medium may include thickeners or particulate fillers such as ceramic particles or carbon-based particles dispersed in a carrier fluid to adjust thermal conduction while maintaining compliance. In some embodiments,the gel may include a silicone gel formed from polydimethylsiloxane with a crosslinker and optional silica thickener, and in some cases the gel may be formulated to remain tacky or to have a low-modulus cured state. In some implementations, a gel may include a polyurethane gel or a styrene-ethylene-butylene-styrene gel in which a mineral oil or synthetic hydrocarbon oil may be immobilized within a polymer network. In some cases, a gel may include a polyacrylamide hydrogel, a polyvinyl alcohol hydrogel, or a gellan-gum hydrogel in which water and a humectant such as glycerin or propylene glycol may be retained to adjust softness while reducing evaporation.

In some embodiments, a wearable heating or cooling device may include an integrated phototherapy subsystem that may deliver red light or near-infrared light to tissue adjacent a bladder or adjacent a contact interface. In some implementations, the phototherapy subsystem may include one or more light-emitting diodes mounted on a flexible substrate positioned between an outer layer and a skin-facing layer, and the light-emitting diodes may emit within one or more wavelength bands such as between about 620 nanometers and about 700 nanometers for red light and between about 780 nanometers and about 950 nanometers for near-infrared light. In some cases, the flexible substrate may be arranged as an array with spacing selected to provide substantially uniform irradiance across a target area, and a diffuser layer, a light guide film, or a reflective backer may be positioned to spread light laterally while limiting stray emission away from the body. In some implementations, the light-emitting diodes may be positioned in regions of the bladder assembly that may remain relatively flat under bending to reduce strain on electrical interconnects, and the interconnects may be routed along serpentine traces to accommodate motion. In some cases, a controller may drive the light-emitting diodes according to a duty cycle program that may include continuous emission or pulsed emission, and the controller may coordinate the program with operation of a pump, a fan or blower, or a thermoelectric element by scheduling light emission during intervals when a measured skin temperature is within a target range.

In some embodiments, a bladder may be implemented as or integrated into a mattress cover or a blanket by forming a multilayer textile laminate that may include a sealed working-liquid layer and one or more comfort layers. In some implementations, the working-liquid layer may be formed as a network of welded or bonded channels between polymer films, and the channel network may be arranged as a serpentine pattern, a grid pattern, or a set of parallel zones that may distribute liquid across a sleeping surface. In some cases, the mattress cover or blanket may include multiple independently supplied zones, and a controller may actuate one or more valves to direct working liquid to a selected zone while restricting flow to other zones based on a user setting or a temperature sensor reading at a respective region. In some implementations, a manifold at an edge of the cover or blanket may couple the channel network to one or more flexible hoses that may connect to a remote conditioning module, and a coupling routine may include engaging a quick-disconnect fitting with an automatic shutoff valve to reduce leakage during removal. In some embodiments, the textile laminate may include an insulating layer positioned on a side opposite the user-facing side to reduce heat exchange with a mattress interior or ambient air, and in some cases a spacer fabric layer may be positioned on the user-facing side to allow compliance and reduce localized pressure points while maintaining thermal coupling between the working liquid and the user.

When assembled, the opposite surface of the sheet 84 relative to the view shown in FIG. 11, that is the surface of the sheet 84 shown in the view of FIG. 10, may be thermally welded, glued, or otherwise sealed around its edges to the opposing face of the base 72 in the region indicated by the dotted lines 88 and 90 shown in FIG. 11, thereby forming a sealed channel 82 by which working fluid may flow in through the inlet 78 and out through the outlet 80 as shown in FIG. 10. This process may involve hot air welding, ultrasonic welding, or thermal compression bonding. Polyurethane-based adhesives may be particularly effective, as they may form strong chemical interactions with both materials. Cyanoacrylate adhesives, epoxy resins, or acrylic-based adhesives may also be employed. Some embodiments may include the use of solvent welding, where a compatible solvent such as tetrahydrofuran (THF) or methyl ethyl ketone (MEK) is applied to dissolve the surface layers of both TPU and EVA. The softened surfaces may then be pressed together, allowing molecular interdiffusion before the solvent evaporates, creating a permanent bond. Other implementations may incorporate pressure-sensitive adhesives (PSAs) or double-sided adhesive tapes designed for flexible material bonding.

In some embodiments the heat exchanger 70 may be flexible, for instance, capable of non-plastic deformation to curve along a curved surface corresponding to portions of a typical adult human body like the knee, back, elbow, shoulder, neck, head, ankle, chest, belly, or the like. In some embodiments the heat exchanger 70 may non-plastically deform to bend around a radius of between 4 and 10 centimeters, like between 5 and 8 centimeters. Some embodiments may be more rigid, for instance, having a bend radius between 10 and 20 centimeters. In some cases, the backside of the foam base 72 may be biased by one of the soft robotic actuators described in the applications incorporated by reference or inflatable bladders or other resilient members, thereby pressing the surface of the sheet 84 against the wearer's body, which as noted may be a human or an animal, including for example a horse.

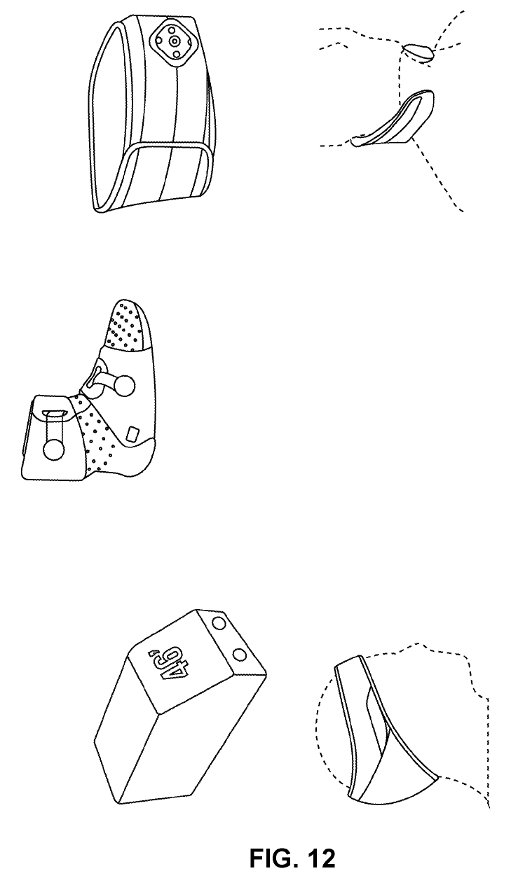

FIG. 12 illustrates various example embodiments of components that may be implemented with the techniques described herein. Examples include the Illustrator Precision Cooler, which may be a desktop or bedside non-portable unit or portable unit. For instance, one that is not wearable but can be plugged into a wall outlet to power the ambient to liquid heat exchanger 12 like that described above or the other example embodiments. Some embodiments may include a wearable thermal brace like that shown. For instance, to brace and provide thermal therapy to an ankle or foot. Some embodiments may include the pain treatment belt like that shown. For instance, like the wearable thermal brace to position and bias the various body liquid heat exchangers described herein against the human body. In another example, a concussive device may bias such a heat exchanger against the wearer's head or a thermoregulation device like that shown may bias such a device against the wearer's neck or head. In some cases, the illustrated techniques may be implemented with inflammation sensing and may be configured to respond to sensed inflammation by modulating temperature or thermal energy into or out of the body.

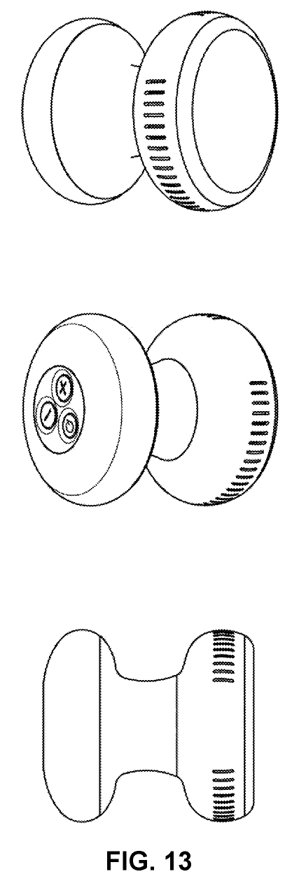

FIG. 13 illustrates an example of a handheld cooling device designed to cool the wearer's palm, for instance, while running. The illustrated device may include a set of buttons on one side to turn it on, increase the temperature or decrease the temperature, and the opposite side may be a metal base, for instance, a Peltier device or adjacent a body liquid heat exchanger like those described above to conduct heat into or out of the wearer's palm. Some embodiments may include an array of apertures to carry ambient air into or out of the illustrated device, for instance, as driven by a fan or blower.

In some embodiments, a wearable heating or cooling device may include a hand interface assembly in which a working liquid may be conducted adjacent a metal plate (e.g., instead of the bladder) positioned to contact a palmar surface of a user’s hand (e.g., with something like the above-described module replicated in a handle with a metal plate in place of the Peltier element, for direct hand contact on that metal plate). In some implementations, the hand interface assembly may be integrated into an insulated glove, mitt, or hand chamber that may include an exterior insulation layer and an interior liner, and in some cases the metal plate may be positioned between the insulation layer and the liner so that the plate may be thermally coupled to a liquid channel while remaining exposed at a contact surface. In some embodiments, a liquid channel may be formed as a serpentine passage adjacent a back side of the metal plate, and in some cases the passage may be defined by a thin metal microchannel plate bonded to the metal plate, by a polymer manifold bonded to the metal plate, or by a welded film bladder pressed against the metal plate. In some implementations, the working liquid may be circulated through the passage by a pump, and a controller may drive the pump to maintain a target liquid temperature while monitoring a temperature sensor embedded in the plate or positioned in the liquid near the plate. In some cases, the glove or chamber may include a cuff seal that may reduce convective exchange with ambient air, and the cuff seal may include an adjustable closure so that the hand chamber may be loosely donned and then tightened to reduce gaps around a wrist. In some implementations, a moisture management layer may be positioned adjacent the liner, and in some cases a desiccant cartridge may be coupled to a recirculated airflow path within the glove to reduce humidity while the plate remains cooled.

In some embodiments, a metal contact surface may be shaped and mounted to encourage consistent contact while allowing a user to insert and remove a hand between exercise intervals. In some implementations, the metal contact surface may be a contoured plate, a curved handle segment, or a knob-shaped element that may be grasped, and in some cases the metal may include aluminum, copper, stainless steel, or a plated alloy. In some embodiments, the metal contact surface may include a textured region, a knurled region, or an elastomer overmold on non-contact portions while a high-conductivity contact region may remain exposed. In some implementations, the metal contact surface may be mounted on a compliant suspension that may allow slight translation when gripped, and in some cases the suspension may include springs or elastomer members that may maintain contact force across different hand sizes. In some embodiments, a quick-disconnect coupling may allow the glove or chamber to be connected to a remote liquid-conditioning module, and in some cases the coupling may include self-sealing valves so that the user may detach the glove without leaking working liquid.

In some embodiments, the hand interface assembly may be integrated with workout equipment so that a user may cool or heat through contact with a conductive element between sets. In some implementations, a conductive, liquid-coupled handle segment may be integrated into a stationary bicycle handlebar, an elliptical trainer handle, a rowing machine handle, a treadmill handrail, or a stepper handle. In some cases, a conductive, liquid-coupled knob or grip may be integrated into a free-weight handle such as a dumbbell handle, a kettlebell handle, a pull-up bar grip, a dip bar grip, or a cable machine attachment such as a straight bar, a rope handle, or a single D-handle. In some implementations, a bench or rack may include a hand-rest station in which a user may insert one or both hands into insulated chambers coupled to the liquid-conditioning module, and in some cases a control routine may detect hand insertion by a pressure sensor or proximity sensor and may increase liquid flow during detected insertion intervals while reducing flow between insertions.

In some embodiments, a road bicycle may include a conductive hand-contact assembly integrated into a handlebar grip or a bar-end element, and a liquid-conditioning module may be powered by an electrical generator integrated into a wheel hub. In some implementations, the hub generator may produce alternating current responsive to wheel rotation, and an onboard power stage may rectify the alternating current, regulate voltage, and charge an energy storage element such as a small battery or supercapacitor so that power may be available during coasting or brief stops. In some cases, the regulated power may drive a pump that may circulate a working liquid through a liquid path routed along the handlebar and adjacent a metal plate or metal liner within a grip, and a controller may modulate pump speed based on a temperature sensor positioned in the working liquid or in the metal plate. In some implementations, the regulated power may also drive a thermoelectric element thermally coupled to the working liquid through a compact liquid-to-solid exchanger mounted near a stem, top tube, or down tube, and the controller may adjust thermoelectric drive current based on a target plate temperature while accounting for available generator power inferred from wheel speed and storage state. In some cases, one or more quick-disconnect couplings may allow the handlebar liquid path to be serviced, and a routing arrangement may include strain relief at bar-tape transitions and flexible sections at steering pivots so that liquid lines may accommodate steering motion.

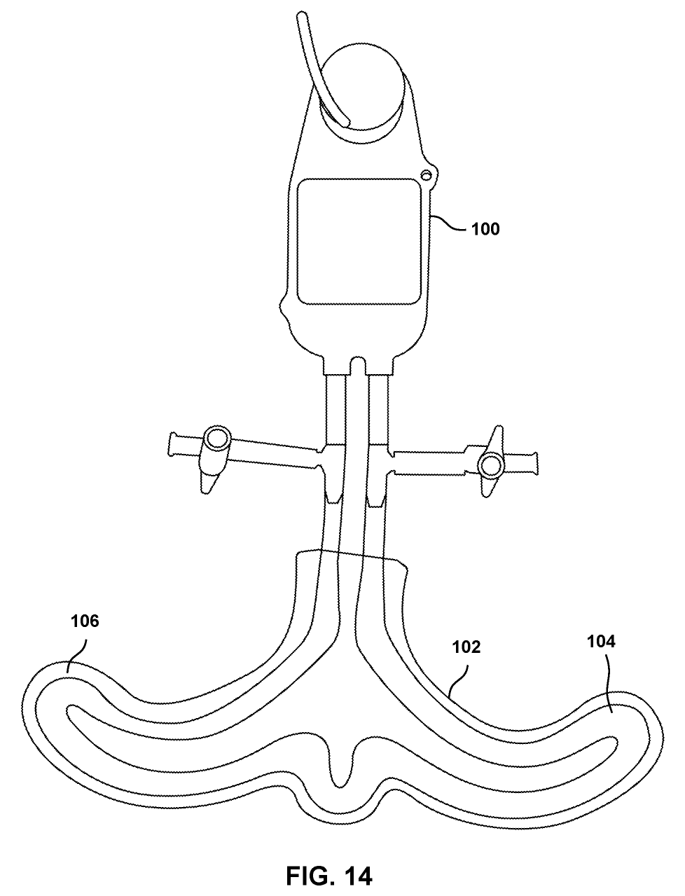

FIG. 14 illustrates an example embodiment configured to apply heat or cooling therapy to a wearer's ankle. In some embodiments, the illustrated ambient to liquid heat exchanger 100 may operate like the ambient liquid heat exchanger 12 described above, and the illustrated body to liquid to heat exchanger 102 may operate like those examples described above. For instance, with the wearer's ankle biased against the middle portion about which the heat exchanger 102 is symmetric, with the left and right wings 104 and 106 wrapped around and biased against the sides of the wearer's ankle.

In some embodiments, a joint brace may include a sleeve that may slide over an arm or leg while the sleeve remains loose, and in some cases the sleeve may carry a bladder that may be positioned at or near a target tissue region once the sleeve is seated. In some implementations, the sleeve may be coupled to a semi-rigid frame that may include an upper segment and a lower segment with a hinge that may allow flexion and extension about an axis that may be set by an adjustable hinge mount. In some cases, a cable routing network may be integrated into the frame, and the routing network may include low-friction guides, pulleys, or lined conduits that may direct a cable along repeatable paths without pinching during bending. In some implementations, the frame and the routing network may be formed as a monolithic structure in which cable channels may be created during molding or additive manufacturing, and in some cases high-strength fibers may be braided or laid into the structure so that the channels may remain stable under tension while the surrounding material may remain compliant. In some embodiments, the cable may be coupled to a rotary dial tensioner that may include a spool and a one-way clutch, and in some cases rotation of the dial may wind the cable onto the spool while the one-way clutch may resist reverse rotation to hold tension.

In some embodiments, a tightening operation may bias the bladder toward the wearer by converting cable tension into circumferential compression of the brace segments. In some implementations, a single cable may be routed through a series of guide points around the upper segment and the lower segment, and in some cases the cable path may include a zig-zag lacing pattern through eyelets or low-profile loops so that cable shortening may draw material inward across a broad area. In some cases, the lacing pattern may be arranged to avoid spanning directly across the hinge line, and in some implementations separate cable loops may be provided for the upper segment and the lower segment while a shared spool may pull both loops through a junction block that may act as a tension splitter. In some embodiments, the junction block may include a differential pulley or a sliding equalizer bar that may redistribute tension between branches based on measured branch travel, and in some cases the equalizer may reduce localized pressure peaks by allowing one branch to take up slack before another branch increases tension. In some implementations, the bladder may be carried on an inner layer that may float relative to an outer structural layer, and in some cases the cable network may pull the outer structural layer inward while the inner layer may translate slightly to center the bladder over the target region. In some embodiments, a release operation may be performed by pulling the dial outward or actuating a release lever that may disengage the one-way clutch, and in some cases a return spring in the spool may pay out cable to return the brace to a slack state that may allow removal.

In some embodiments, the brace may allow elbow bending while maintaining bladder contact by maintaining tension in regions above and below the joint rather than relying on high compression directly across a crease line. In some implementations, the hinge may include an overlapping shell geometry that may slide during flexion, and in some cases the cable routing may traverse the overlap through rolling guides that may maintain cable length while the hinge angle changes. In some embodiments, a bladder-facing surface may include a low-friction liner that may allow the bladder to shear slightly against the skin during motion while the brace remains tensioned, and in some cases the bladder may include internal baffles that may reduce migration of working liquid when the elbow flexes. In some implementations, a motion-constraining embodiment may include adjustable hard stops that may be repositioned to set a flexion limit or an extension limit, and in some cases the stops may be implemented as removable pins, threaded limit screws, or cam blocks that may engage a stop surface on the hinge. In some embodiments, a protective embodiment may include a damping element that may resist rapid motion, and in some cases the damping element may be a friction clutch at the hinge, a viscous dashpot, or an elastomer element that may deform under load to slow rotation. In some implementations, a tightening limit may be implemented by a torque-limiting clutch in the dial tensioner, and in some cases the clutch may slip above a threshold torque while the spool rotation may continue to provide tactile feedback without further increasing strap tension.

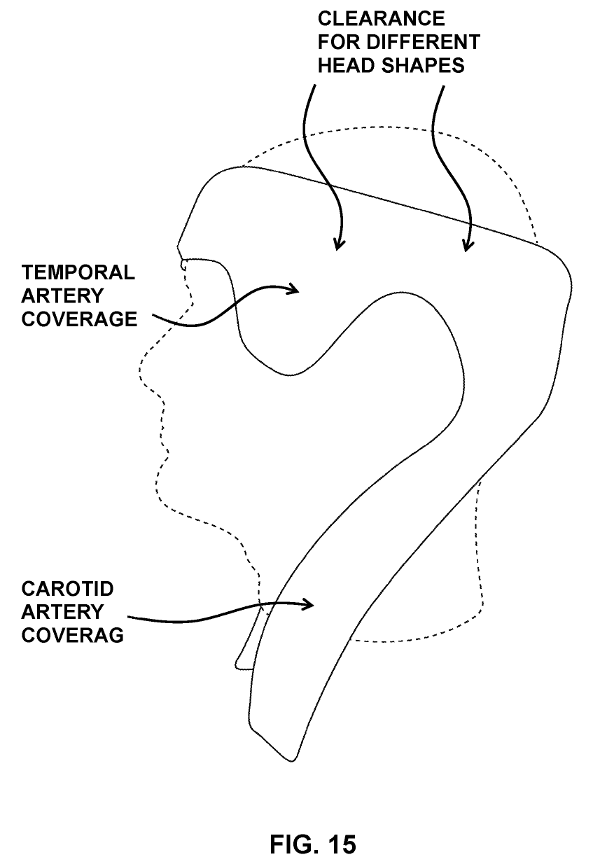

FIG. 15 illustrates an example of a head-worn device configured to wrap around a wearer's head and bias a body-to-liquid heat exchanger like those described above against various portions of the wearer's head and neck, as illustrated. In some embodiments, tubes carrying a working fluid to or from an ambient to liquid heat exchanger may extend from the lower portion of the neck flaps illustrated to carry the working fluid to or from that heat exchanger thereby keeping the weight of that ambient to liquid heat exchanger off the wearer's head and mounted more comfortably, for example on the wearer's body like their chest or waist, for instance, with a strap or clip or in one of the below described vests.

In some embodiments, a head-worn wearable heating or cooling device may be implemented as a flexible assembly that may be positioned around a head of an organism as a band, cap, or partial cap, and in some cases a body-contact portion may be positioned at one or more regions that may include a forehead, a temple region, a crown region, an occipital region, a suboccipital region, or a region adjacent an ear. In some implementations, the body-contact portion may include a bladder that may define part of a working liquid flow path and that may be shaped to follow head curvature, and in some cases the bladder may be segmented into multiple zones that may be coupled by a manifold so that a controller may vary flow distribution among zones. In some embodiments, a wearable carrier may include a compliant frame that may define a perimeter band and one or more cross straps, and in some cases the carrier may include a low-profile adjustment mechanism that may be tightened after the carrier is placed on the head. In some implementations, a tightening sequence may include placing the carrier loosely, aligning the bladder to a target contact region by sliding the carrier relative to hair, and then increasing tension until a contact pressure sensor or an inferred contact quality metric indicates a target range. In some cases, the carrier may include one or more comfort layers such as a spacer fabric adjacent skin, and in some implementations the spacer fabric may be selected to permit moisture transport while a liquid barrier film may remain sealed.

In some embodiments, a head-worn bladder may be constructed as a multilayer laminate that may include a liquid boundary layer, a reinforcement layer, and a skin-contact layer, and in some cases seams may be formed by radio-frequency welding, heat sealing, ultrasonic welding, adhesive bonding, or another bonding process. In some implementations, the bladder may include internal baffles or internal inserts that may reduce ballooning and that may maintain a defined channel height under strap tension, and in some cases the inserts may define flow-distribution regions that may spread working liquid across a broad contact area. In some embodiments, a channel layout may include one or more serpentine paths that may traverse a forehead region while avoiding a hairline region, and in some cases a branching layout may feed a left side and a right side via separate branches that may be balanced by orifices, restrictors, or flow control valves. In some implementations, the bladder may include one or more compliant hinges that may allow the assembly to flex during facial movement or head tilt, and in some cases hinge regions may be formed by reducing laminate thickness or by including fold lines that may be aligned with expected bending axes. In some embodiments, a leak-detection feature may be included by placing one or more conductivity traces or humidity sensors adjacent the bladder, and in some cases a controller may reduce pump output or thermoelectric drive current responsive to a detected leak signature.

In some embodiments, a remote thermal module may be worn off the head while remaining coupled to the head-worn bladder by one or more flexible tubes, and in some implementations the remote thermal module may be positioned at a collar, shoulder, chest, back, or waist location. In some cases, a routing approach may include running tubes along a garment seam or under a collar while allowing head rotation, and in some implementations a strain-relief feature may be formed by anchoring a tube at a pivot region and providing a flexible loop that may accommodate yaw rotation without kinking. In some embodiments, a quick-disconnect coupling may be placed at a collar interface, and in some cases the quick-disconnect coupling may include automatic shutoff valves so that separation may occur without releasing working liquid. In some implementations, the remote thermal module may include a pump, a thermoelectric element, and one or more heat exchangers, and in some cases the thermoelectric element may be driven to set a working liquid temperature while the pump may circulate the working liquid through the head-worn bladder and back to the remote thermal module. In some cases, a controller may coordinate pump flow rate with thermoelectric drive current, and in some implementations the controller may apply a control routine that may include reading a skin-side temperature sensor, reading a working liquid inlet temperature and outlet temperature, estimating a thermal response, and adjusting setpoints to maintain a target temperature at the head-worn contact region.

In some embodiments, a head-worn implementation may include airflow management adjacent the bladder while maintaining net heating or cooling delivered by the working liquid. In some implementations, a microperforated liner may be positioned between skin and the bladder, and in some cases a low-flow air path may deliver recirculated air through an array of openings to refresh near-skin air without introducing a large amount of ambient air at a different temperature. In some embodiments, a recirculation loop may include a small blower and a desiccant cartridge positioned in a duct, and in some cases the desiccant cartridge may reduce humidity of recirculated air to reduce condensation risk on cooled surfaces. In some implementations, a controller may estimate a dew point based on measured temperature and humidity and may adjust airflow rate, recirculation ratio, or working liquid temperature when the estimate indicates that condensation may occur. In some cases, airflow may be provided in pulses, and in some implementations a pulse schedule may be selected based on an activity state inferred from inertial sensor data so that airflow may increase during movement and decrease during stillness. In some embodiments, an edge seal may bound a shallow air cavity adjacent the bladder, and in some cases the edge seal may be segmented to accommodate hair and contour changes while maintaining a defined leakage rate.

In some embodiments, a head-worn carrier may be arranged to maintain comfort and stability while permitting bending and rotation of the neck. In some implementations, the carrier may include a rear cradle that may rest at an occipital region and a front band that may rest at a forehead region, and in some cases a cross strap may distribute load across a crown region. In some embodiments, an adjustment mechanism may include a dial-driven spool that may retract one or more cables routed through low-friction guides, and in some cases cable tension may be distributed using a splitter block so that a left-side branch and a right-side branch may tighten substantially together. In some implementations, the carrier may include a quick-release feature that may be actuated by pulling a tab to reduce tension, and in some cases the quick-release feature may disengage a one-way clutch to permit rapid loosening. In some embodiments, a rigid or semi-rigid element may be positioned to protect a thermally active region from impact, and in some cases the rigid element may be isolated from skin by a compliant layer so that contact pressure may remain distributed.

In some embodiments, a head-worn wearable heating or cooling device may be integrated with a helmet, a virtual reality display, or an augmented reality display by coupling a liquid-circulating bladder and associated routing features to an interior padding system. In some implementations, a bladder may be laminated into or placed behind a comfort liner that may contact a forehead region, a temple region, an occipital region, or a crown region, and the bladder may define a working liquid flow path that may be fed by one or more flexible tubes routed through channels in the helmet shell or through a headband structure of the display. In some cases, the channels may include strain-relief loops positioned near hinge points of an adjustable headband so that tube curvature may remain within a bend-radius constraint during headband tightening and during head motion. In some implementations, a manifold may distribute working liquid to multiple zones within the liner, and a controller may actuate one or more valves to adjust flow among zones based on temperature sensors positioned in the liner or in the working liquid. In some embodiments, a remote thermal module may be mounted on a rear strap, a counterweight module of the display, or a belt-worn pack, and the remote thermal module may include a pump and a conditioning stage that may set working liquid temperature while the controller may coordinate pump output with measured skin-side temperature. In some cases, electrical conductors for sensors and optional light-emitting diode arrays may be routed along the same channels as the tubes using flexible printed conductors, and in some implementations connectors at a removable liner interface may include self-sealing fluid couplers and spring contacts that may allow the liner to be detached for cleaning or replacement.

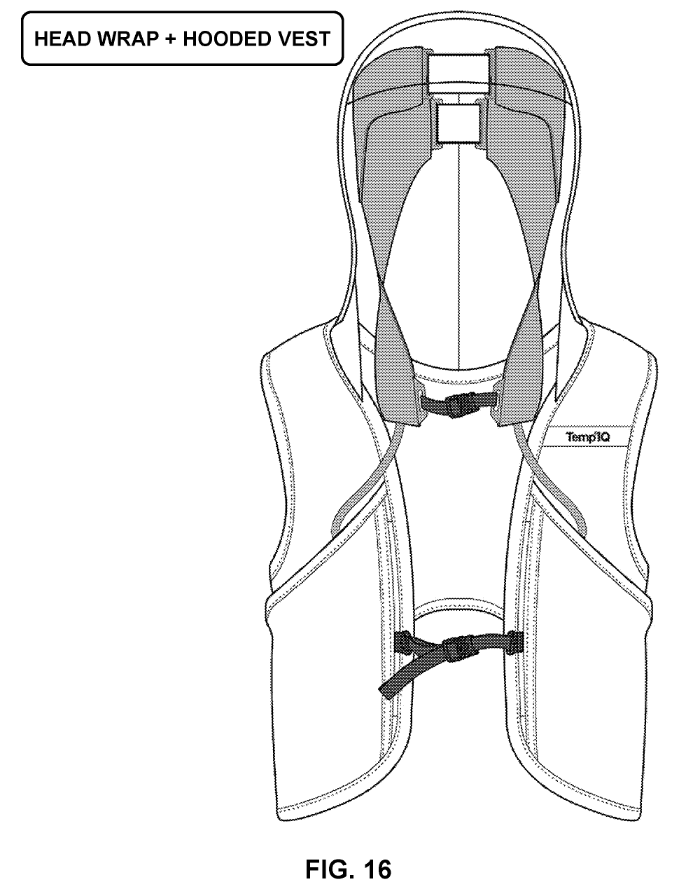

FIG. 16 illustrates an example of such a vest paired with the head-mounted device shown in FIG. 15 from a front view. As illustrated, tubes may carry a working fluid into pockets in the illustrated vest, which in some cases may include passages for the tubes to extend around the back or to otherwise fluidly communicate with an ambient to liquid heat exchanger forming a circulatory loop. In some cases, the illustrated vest in FIG. 16 may also include other body to liquid heat exchangers biased by the vest against the wearer's body, for instance, the wearer's chest, shoulders, or back.

In some embodiments, a wearable thermal garment may include multiple independently controllable thermal zones distributed across a body region, such as a vest spanning a chest and back, a wrap spanning an arm or leg, or a boot spanning a foot and calf. In some implementations, each zone may be defined by a corresponding liquid channel network positioned adjacent a target tissue region, and the channel networks may be arranged so that a first zone may preferentially couple to a first anatomical region while a second zone may preferentially couple to a second anatomical region. In some cases, a vest may include separate zones for an upper back, a lower back, a left pectoral region, and a right pectoral region, and a boot may include separate zones for an ankle region, an arch region, a heel region, and a calf region. In some implementations, a wrap may include separate zones for forearm flexors, forearm extensors, biceps, triceps, quadriceps, hamstrings, or a region adjacent a tendon insertion. In some cases, the garment may support a thermal “map” by storing zone identifiers and zone geometry in a data structure that associates each zone with a spatial region, a set of sensors, and one or more control parameters.

In some embodiments, zone separation may be achieved using a micro-manifold and valving system rather than a single monolithic bladder. In some implementations, a central supply header and a central return header may be routed along a seam or edge of the garment, and branch passages may extend from the headers into respective zones. In some cases, the branch passages may be formed as laminated microfluidic channels in a flexible film stack, molded channels in a polymer strip, or embedded tubing routed through stitched channels. In some implementations, a micro-manifold may include a distributed block that provides multiple branch ports at a compact location, and the block may integrate seals, flow restrictors, and valve seats in a single body. In some cases, the valves may be arranged as normally-closed micro-valves positioned at or near each branch port, such that opening a valve may allow working liquid to flow through a corresponding zone while closing the valve may reduce or prevent flow through that zone. In some implementations, the valves may include solenoid valves, latching valves, piezoelectric valves, shape-memory actuated pinch valves, or microfluidic membrane valves formed by deforming an elastomer membrane against a valve seat. In some embodiments, a valve and manifold assembly may be arranged to remain flexible by distributing valves across multiple small modules connected by flexible interconnects, rather than concentrating all valves in a single rigid block.

In some embodiments, zone-level control may be performed by regulating at least one of (i) flow rate through a zone, (ii) mixing ratio of supply streams, or (iii) a zone-specific supply temperature. In some implementations, a controller may regulate flow rate by modulating valve duty cycle using pulse-width control, by commanding proportional valves to intermediate openings, or by selecting among multiple fixed restrictors using a valve tree. In some cases, a pump may maintain a base circulation through the supply and return headers while valves allocate that circulation among zones. In some implementations, a mixing architecture may provide two working-liquid streams at different temperatures, and a zone manifold may include a mixing junction in which a first valve meters a fraction of a warmer stream and a second valve meters a fraction of a cooler stream to achieve a zone supply temperature. In some cases, a zone may include a bypass loop and a heat-exchange loop, and the controller may adjust a bypass valve to vary how much liquid passes through a thermally active portion of the zone versus bypassing it. In some embodiments, the garment may include check valves or flow-balancing features so that closing one zone may not induce backflow or unintended flow increases through another zone.

In some embodiments, the garment may include sensors that support independent regulation of zones and support updating a thermal map over time. In some implementations, sensors may include liquid temperature sensors at one or more points on a supply header or branch line, liquid temperature sensors at one or more return branch lines, skin-side temperature sensors embedded in a liner near each zone, humidity sensors near zones prone to condensation, and pressure or flow sensors that estimate zone flow. In some cases, the controller may estimate zone heat transfer by combining measured flow with a measured temperature difference between zone inlet and zone outlet, and the controller may adjust a valve opening to drive an estimated heat transfer toward a target. In some implementations, the controller may compensate for contact variability by using contact pressure sensors or by inferring contact quality from a thermal response to a small control perturbation, and the controller may reallocate flow among zones when contact quality is reduced in a zone. In some embodiments, a calibration routine may be performed at startup by sequentially opening one zone at a time, measuring a corresponding flow or pressure response, and storing valve-to-flow coefficients for later control.

In some embodiments, zone control may coordinate with garment form factor constraints, such as bending at a joint or compression changes during muscle movement. In some implementations, a boot may route micro-manifolds along a shin region and route branch lines around an ankle using strain-relief loops so that repeated dorsiflexion does not kink lines. In some cases, a knee or elbow wrap may position manifolds away from a hinge line and may route branches through flexible hinge regions using flattened tubing or laminated channels with widened radii. In some embodiments, a vest may position manifolds near side seams and may route branches across the back using multiple parallel thin channels to reduce bulk while preserving flexibility. In some implementations, the controller may alter zone setpoints based on posture or motion inferred from inertial sensors, such as reducing flow through a region that is compressed by a seated posture and increasing flow through a region that remains in contact. In some implementations, a user interface may allow selection of a thermal program that specifies zone setpoints and time schedules, and the controller may execute the program by updating valve commands, pump output, and thermal conditioning output while maintaining independence among zones.

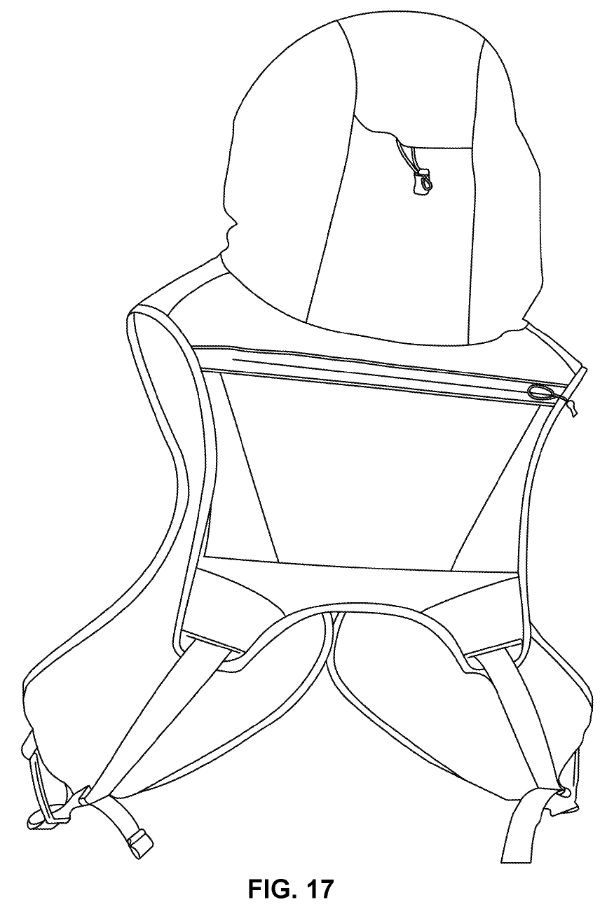

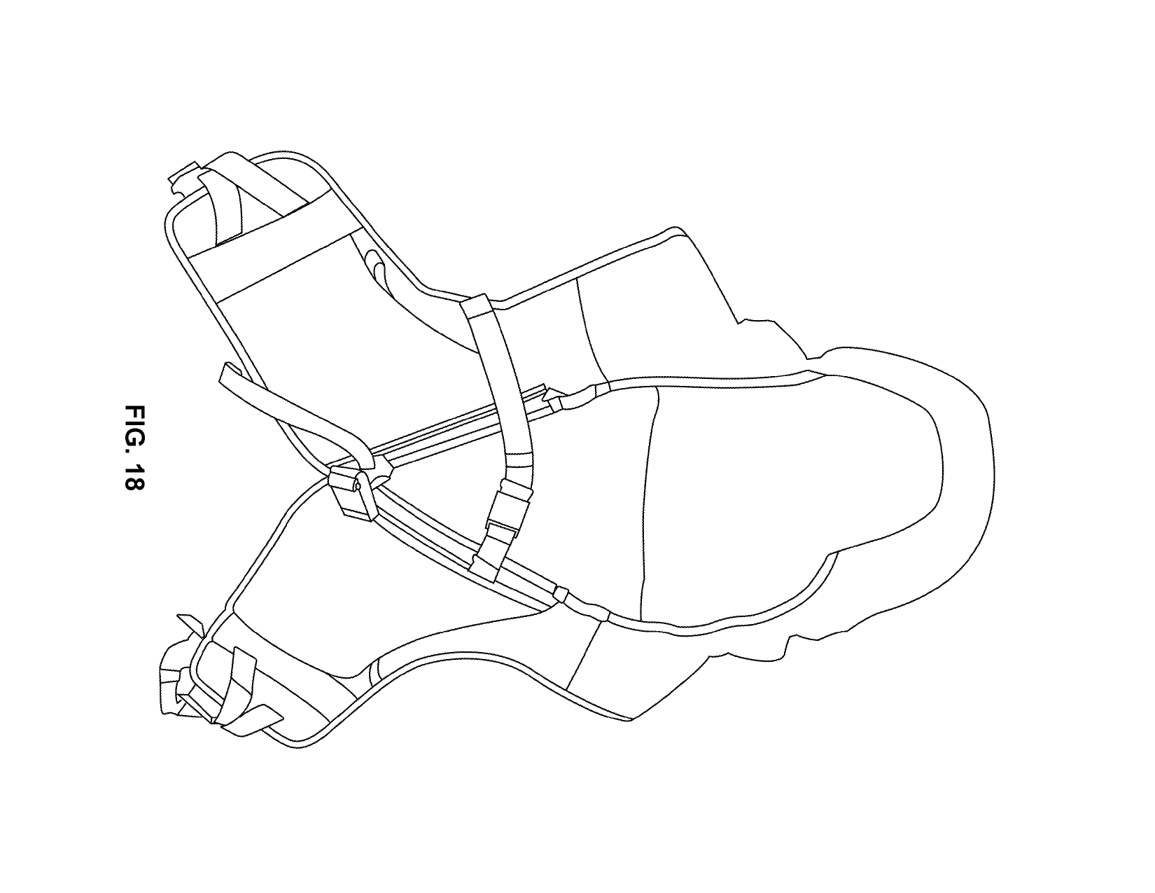

FIG. 17 is an example view of a back of a vest with a hood configured to carry such a set of heat exchangers like those described above. And FIG. 18 is a front view of that embodiment. In some cases, the vest may include a chest strap like that shown, and the vest may include pockets or other cavities for holding the various heat exchangers shown above with routing for tubing to bias body to liquid heat exchangers against the human's body. Similar techniques may be used to apply such heat exchangers to other parts of the human body or parts of animal bodies, for instance, a horse's back, neck, belly, legs, or the like. Similar techniques may be used for corresponding portions on a dog, cat, or other animal.

In some embodiments, a wearable heating or cooling device may be implemented as a vest that may position a liquid-circulating bladder across one or more torso regions while a conditioning module may be carried on the vest in a manner that allows walking and bending. In some implementations, the vest may include a front panel and a back panel joined by shoulder straps and side straps, and a bladder may be laminated into one or both panels as a sealed network of welded channels that may define part of a working liquid flow path. In some cases, a manifold may be positioned near a side seam or lower hem, and flexible tubes may route working liquid between the manifold and a conditioning module mounted on a rear panel, a side panel, or a belt interface coupled to the vest. In some implementations, the vest may include multiple independently supplied zones such that a first zone may cover an upper back and a second zone may cover a lower back or a chest region, and a controller may actuate one or more valves to adjust flow among the zones based on temperature sensors positioned in the working liquid, at a skin-facing liner, or both. In some cases, the vest may include a tensioning mechanism that may adjust contact pressure by retracting one or more cables through low-friction guides, and a control routine may incrementally tighten the vest after donning by commanding a spool to take up slack, measuring strap tension or contact pressure, and stopping when a target range is reached.

In some embodiments, a battery carried by the vest may be charged by inductive power transfer when a user sits in a seat such as a driver’s seat. In some implementations, the vest may include a receive coil positioned in a back panel or a seat-contact panel, and the seat may include a transmit coil embedded under upholstery, where the receive coil and transmit coil may be arranged to magnetically couple when the user’s back or torso presses into the seat. In some cases, the receive coil may be implemented as a flexible litz-wire winding or a printed spiral on a flexible substrate, and the receive coil may be backed by a ferrite sheet or a laminated magnetic composite to shape the magnetic field and reduce coupling to other conductors in the vest. In some implementations, the seat may include multiple transmit coils arranged as an array, and a controller in the seat may select an active transmit coil based on measured impedance changes that may indicate which coil is most closely aligned with the receive coil. In some cases, the vest may include one or more alignment features such as low-profile magnets, passive resonant markers, or a shaped cushion interface, and an alignment routine may determine coupling quality by measuring received voltage, coil current, or a resonant frequency shift.

In some embodiments, a charging session may be controlled by a power-management routine that may coordinate a rectifier, a regulator, and a battery management circuit within the vest. In some implementations, a communication exchange may occur between seat electronics and vest electronics using load modulation on the inductive link or a separate short-range radio, and a negotiated power level may be set based on battery state, thermal conditions, and seat power limits. In some cases, the charging routine may begin at a low power level, may measure temperature of a receive-coil region and a battery pack, and may ramp charging current while keeping measured values within thresholds. In some implementations, the system may perform foreign-object detection by monitoring transmit-coil losses, may reduce power when losses exceed a threshold, and may pause charging when a conductive object is detected in the field. In some cases, the vest controller may schedule high-load thermal operation to avoid coinciding with peak charging intervals by reducing thermoelectric drive current, adjusting pump flow, or operating a fan or blower at a reduced duty cycle while charging, and the vest controller may resume a prior thermal program after charging power falls below a threshold or after the user exits the seat.

In some cases, a heat exchanger like those show in in FIGS. 2-9 may function as a “Central Heat Port," wherein one central dissipation unit dissipates localized TEC-exchangers via the same heat pipe couplers to a single exhaust port or liquid exchanger.

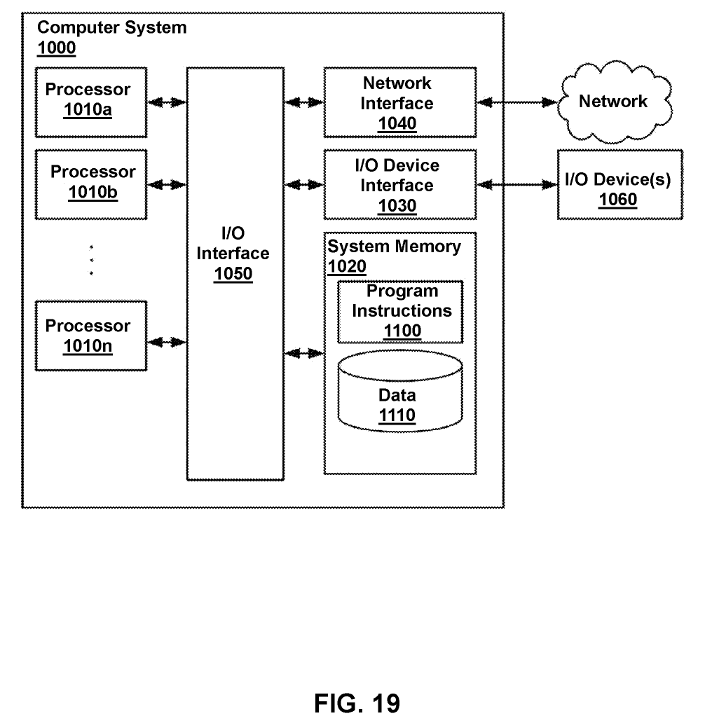

In some cases, as described in the patent filings incorporated by reference, thermal flux driven by the systems above may be controlled by software executed on a computer system, such as the one in FIG. 19, like an embedded system, a mobile device, or a desktop device. In some cases, this software may receive sensor data (e.g., from thermocouples on the body of the user, in the working fluid, or on or at the various heat exchangers) and modulate heat flux in response (e.g., by reversing and increasing or decreasing current to the Peltier elements, modulating fluid flow by changing pump speed, changing fan speed, or the like). An "Energy Value" may be computed by software, the electronic heat exchanger, and/or various sensors such as HRV. This may be done with precision bladder designs on localized body, in conjunction with ambient data, to make the bladder or heat exchanger a sensor by assigning an energy value to the user area. Some embodiments may use this value (or sensed temperatures) to automatically turn off thermal flux at values above or below respective thresholds. Some embodiments may use the energy value for biodata, such as detecting inflammation, signs of infection, or heat exhaustion, e.g., by sensing how much power is consumed to hold a target temperature, an amount of heat generated by the user’s body may be inferred, thereby potentially indicating such phenomena. In effect, the wattage introduced by the user to the system may be calculated with AI-driven insights in some embodiments.

In some embodiments, a wearable heating or cooling device may be arranged as multiple subassemblies that may be worn at different body locations while remaining coupled by flexible fluid lines, electrical conductors, or both. In some implementations, a body-contact subassembly may be worn at a neck, head, torso, wrist, ankle, or joint location, and a remote subassembly may be worn at a waist, chest, back, thigh, or shoulder location. In some cases, a routing routine may select a path for one or more tubes based on one or more constraints that may include minimum bend radius, avoidance of pinch points during flexion, and avoidance of pressure points under straps. In some implementations, strain-relief anchors may be placed at selected locations, and a tube jacket may be overmolded with a compliant polymer at the anchors while leaving a low-friction outer surface along a sliding region to allow relative motion between the tube and a garment channel.

In some embodiments, a device may include a mechanically robust quick-attach interface between a wearable carrier and a thermal module. In some implementations, a latch may be operated by rotating a lever through a defined range, and the lever rotation may drive a cam that may draw the module into a repeatable seated position. In some cases, a seating sequence may include aligning keyed features, advancing the module until a detent may engage, rotating the lever to compress one or more gaskets, and verifying engagement by reading a latch sensor or by measuring a change in electrical continuity across contacts. In some implementations, a fluid coupling may be integrated with the latch, and the latch motion may open one or more spring-biased valves while a seal may remain closed when the module is removed.

In some embodiments, the body-contact portion may include a compliant mechanical frame that may maintain contact while allowing motion. In some implementations, a frame may include one or more compliant mechanisms formed as flexures, living hinges, or spring members, and in some cases the mechanisms may be tuned by selecting thickness, curvature, and material modulus. In some embodiments, a joint-worn form factor may include a multi-link brace that may track a joint axis, and in some cases the brace may include a floating linkage or a sliding interface that may allow small misalignments between the brace axis and an anatomical joint axis during motion. In some implementations, a tightening operation may be performed by a ratcheting spool, a worm-drive spool, or a magnetic clutch spool, and a controller may incrementally tighten the strap by commanding a motor to rotate a defined number of steps, reading strap tension, comparing the measured tension to a target profile, and repeating until the profile may be met.

In some embodiments, the bladder may include internal structures that may manage both flow distribution and shape stability while remaining flexible. In some implementations, an internal insert may be formed as a three-dimensional lattice that may include flow channels and support ribs, and in some cases the insert may be bonded to one or more bladder layers at discrete points to define local channel heights. In some implementations, a manufacturing process may include placing the insert between film layers, welding a perimeter seam, welding one or more internal seam lines, and then performing a pressure test while monitoring for a decay rate. In some cases, the insert may include a porous region that may distribute flow across an area, and in some implementations the porous region may be coated with a thermally conductive coating, a chemically resistant coating, or both. In some embodiments, a bladder may include segmented micro-bladders coupled to a manifold, and in some cases a controller may actuate micro-valves to direct liquid through selected segments based on sensor readings that may include temperature or contact pressure.

In some embodiments, a contact interface may include a layer that may adjust compliance or thickness during operation. In some implementations, an electroactive polymer layer may be driven by a control waveform to change thickness, and in some cases the control waveform may be timed with pump operation to reduce pressure pulsations felt by the wearer. In some embodiments, a pneumatic layer may be filled by a micro-pump, and in some cases the micro-pump may inflate one or more zones while pressure sensors may provide feedback to maintain a target contact pressure. In some implementations, a pneumatic actuator may maintain contact by cycling through a sequence that may include inflating to a first pressure, holding while temperature approaches a setpoint, and deflating to a second pressure during higher-motion periods inferred from inertial sensor readings.

In some embodiments, the heat rejection portion may be arranged to distribute heat across a larger area and to manage airflow with a controlled duct network. In some implementations, a heat sink may be formed as a lattice that may define air passages, and in some cases the lattice may have a gyroid geometry or another triply periodic minimal surface geometry. In some implementations, a conformal coating may be applied to the lattice by electroless plating, physical vapor deposition, atomic layer deposition, or another deposition process, and in some cases a masking and unmasking sequence may preserve selected airflow passages. In some embodiments, a heat sink may include a shroud with adjustable apertures, and in some cases a controller may open or close an aperture based on a temperature gradient measured across a sensor array distributed along the heat sink.

In some embodiments, airflow generation may be implemented in a manner that may reduce thickness or reduce rotating mass at a location worn close to the body. In some implementations, airflow may be generated by a microblower array, and in some cases a controller may activate subsets of microblowers in a rotating schedule based on a duty-cycle plan. In some embodiments, airflow may be generated by an electrohydrodynamic device that may apply a potential between an emitter and a collector, and in some cases the controller may adjust the potential based on humidity, particulate loading, or proximity sensing. In some implementations, an intake path may include a particle trap or filter that may be electrically actuated, and in some cases a maintenance routine may apply a cleaning pulse, detect a change in pressure drop, and then command a brief reverse-flow purge.

In some embodiments, coolant circulation may be provided by a pump architecture that may reduce sealing complexity or that may allow compliance in flexing regions. In some implementations, a pump may be a piezoelectric diaphragm pump driven by a waveform, and in some cases the waveform may be adjusted by varying frequency, duty cycle, or amplitude while monitoring flow. In some embodiments, a pump may be a peristaltic pump integrated into a strap path, and in some cases strap motion may drive rollers to compress a tube segment while a controller may account for strap motion by estimating effective flow from inertial measurements and pressure measurements. In some implementations, a magnetic coupling may drive an impeller without a rotating shaft seal, and in some cases a control routine may detect air bubbles by monitoring a pressure oscillation signature and may perform a purge cycle by increasing flow, opening a bleed valve, or both.

In some embodiments, the device may include distributed sensing and control routines that may adjust operation while a wearer moves through different postures and activities. In some implementations, sensors may include temperature sensors placed in the working liquid at one or more points, skin-side temperature sensors, humidity sensors, contact pressure sensors, and flow sensors. In some cases, a processing routine may time-align sensor samples, filter motion artifacts, and compute a contact quality metric, and in some implementations the contact quality metric may be used to adjust pump speed, fan speed, or thermoelectric drive current. In some embodiments, a controller may perform a system identification routine during a startup or during periodic recalibration, and in some cases the routine may apply a sequence of small control perturbations, measure a thermal response, and update a model that may be used to predict a future temperature trajectory under candidate control actions.

In some embodiments, a power and packaging approach may distribute weight and stiffness while allowing removal and replacement of consumable or serviceable parts. In some implementations, a battery may be arranged as multiple modules embedded in a belt, strap, or garment panel, and in some cases a battery management routine may estimate state of charge by combining coulomb counting with impedance-based estimation and temperature compensation. In some embodiments, a device may include an energy storage element in a textile region, and in some cases a controller may charge the storage element during low-load periods and discharge it during high-load ramps. In some implementations, electronic assemblies may be protected by conformal coatings and by internal seals, and in some cases a diagnostic routine may spin a fan, actuate a pump, measure current draw, measure flow response, and reduce or disable thermoelectric drive when measured values fall outside threshold.

In some embodiments, mass reduction may be achieved by forming structural bodies such as a tank body, a housing, a manifold, or a module shell as thin-walled shells with local reinforcement rather than as solid sections. In some implementations, a polymer housing may be formed as a ribbed shell in which a wall thickness may be reduced while stiffness may be maintained by molded ribs, corrugations, or isogrid patterns, and in some cases the polymer may include long-fiber reinforcement such as glass fiber or carbon fiber in a polyamide, polyphenylene sulfide, or polyether ether ketone matrix. In some implementations, a tank body may be formed from a multilayer film or blow-molded shell in which an inner barrier layer may be combined with an outer impact layer, and in some cases the shell may be locally thickened only at bosses, ports, or latch interfaces using insert molding or overmolding. In some embodiments, a structural element may be formed from a sandwich panel in which thin skins may be bonded to a lightweight core, and in some cases the core may be a polymer honeycomb, a microcellular foam, or a lattice core that may be created by additive manufacturing or by thermoforming a patterned sheet. In some implementations, fasteners may be reduced by forming snap features, compliant latches, or ultrasonic-weld seams, and in some cases welded seams may be placed along low-stress regions while load-bearing regions may include embedded metal inserts that may be localized to threads and bearing surfaces.

In some embodiments, heat sink mass may be reduced by increasing surface area per unit mass using thin fins, lattice geometries, or hollow structures. In some implementations, a heat sink may be formed as an additively manufactured lattice such as a gyroid or another triply periodic minimal surface geometry, and in some cases the lattice may be printed in aluminum or titanium and then coated with copper, nickel, or another thermally conductive layer in selected regions where contact resistance may be reduced. In some embodiments, a heat sink may be formed from folded thin sheet metal, and in some cases a fin stack may be made by stamping, folding, and brazing aluminum sheets to form channels and louvers while reducing bulk metal volume. In some implementations, a heat sink may be made by extrusion or skiving to produce thin, closely spaced fins, and in some cases a portion of the heat sink may be hollowed by machining or by forming a shell around a cavity that may serve as a duct. In some embodiments, a vapor chamber or heat pipe may be used to spread heat to a larger but thinner fin field, and in some cases a heat pipe may be flattened by rolling or pressing to reduce thickness while maintaining a wick structure. In some implementations, a heat sink may include a polymer or composite base with embedded metal foils or embedded graphite sheets, and in some cases pyrolytic graphite, expanded graphite, or graphite fiber sheets may be laminated into the base to spread heat laterally while reducing dense metal mass.

In some embodiments, dense components such as pumps, fittings, and batteries may be mass-reduced by integrating functions into fewer parts and by selecting low-density materials where thermal or mechanical constraints allow. In some implementations, fluid fittings may be formed as polymer quick-connects with integrated valves, and in some cases the fittings may be molded from glass-filled polymers while sealing may be performed by molded elastomer lips rather than metal springs. In some embodiments, a pump may be implemented as a piezoelectric diaphragm pump with a thin pump chamber formed by film lamination, and in some cases a manifold may be formed as a flexible printed circuit substrate with bonded microfluidic layers to integrate sensor wiring and flow channels. In some implementations, energy storage may be distributed across a belt or strap using multiple smaller battery modules, and in some cases the modules may be packaged in thin pouches with local reinforcement rather than in rigid cases. In some embodiments, a counterweight strategy may be replaced by mass relocation, and a mounting structure may position heavier modules close to the torso while a neck, head, or limb contact assembly may remain primarily a bladder, a thin interface layer, and lightweight sensor layers formed from flexible substrates.

FIG. 19 is a diagram that illustrates an exemplary computing system 1000 by which some embodiments may be controlled. A single computing device is shown, but some embodiments of a computer system may include multiple computing devices that communicate over a network, for instance in the course of collectively executing various parts of a distributed application. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term "medium," the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or "a element" includes a combination of two or more elements, notwithstanding use of other terms and phrases for one or more elements, such as “one or more.” The term "or" is, unless indicated otherwise, non-exclusive, i.e., encompassing both "and" and "or." Terms describing conditional relationships, e.g., "in response to X, Y," "upon X, Y,", “if X, Y,” "when X, Y," and the like, encompass causal relationships in which the antecedent is a necessary causal condition, the antecedent is a sufficient causal condition, or the antecedent is a contributory causal condition of the consequent, e.g., "state X occurs upon condition Y obtaining" is generic to "X occurs solely upon Y" and "X occurs upon Y and Z." Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X’ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like "parallel," "perpendicular/orthogonal," “square”, “cylindrical,” and the like, should be construed as encompassing items that substantially embody the properties of the geometric construct, e.g., reference to "parallel" surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms "first", "second", "third," “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:

Embodiment 1. A wearable heating or cooling device, comprising, a bladder configured to be positioned adjacent an organism, the bladder defining part of a working liquid flow path, and an air-to-liquid heat exchanger fluidically coupled to the bladder, the air-to-liquid heat exchanger comprising, a pump configured to circulate a working liquid through the working liquid flow path, a body made of a polymer, a first metal heat exchanger having a plurality of protrusions positioned in a portion of the working liquid flow path inside the body, a Peltier element having a first side adjacent the first metal heat exchanger and a second side opposite the first side, a fan or blower coupled to the air-to-liquid heat exchanger and configured to force air along an air flow path, and a second metal heat exchanger adjacent the second side of the Peltier element, the second metal heat exchanger having a plurality of protrusions positioned in the air flow path of the fan or blower.

Embodiment 2. The wearable heating or cooling device of embodiment 1, wherein the bladder comprises a pair of polymer sheets sealed to one another to define a serpentine liquid channel forming at least a portion of the working liquid flow path.

Embodiment 3. The wearable heating or cooling device of embodiment 1, wherein the bladder comprises a thermoplastic polyurethane film and a textile-backed outer layer configured to be positioned against the organism.

Embodiment 4. The wearable heating or cooling device of embodiment 1, wherein the bladder comprises a multi-layer laminate including an inner barrier layer configured to reduce permeation of the working liquid and an outer layer configured to be heat-sealable.

Embodiment 5. The wearable heating or cooling device of embodiment 1, wherein the bladder includes one or more internal inserts positioned within the bladder to define at least one of (i) a flow-distribution region, or (ii) a baffle region that inhibits ballooning of the bladder.

Embodiment 6. The wearable heating or cooling device of embodiment 1, wherein the first metal heat exchanger comprises aluminum and the plurality of protrusions comprises pins or posts integrally formed with the first metal heat exchanger.

Embodiment 7. The wearable heating or cooling device of embodiment 1, wherein the second metal heat exchanger comprises copper and the plurality of protrusions comprises fins configured to promote turbulent airflow in the air flow path.

Embodiment 8. The wearable heating or cooling device of embodiment 1, wherein the bladder is configured as a neck-wearable band having a curved profile that wraps at least partially around a neck of the organism.

Embodiment 9. The wearable heating or cooling device of embodiment 1, wherein the bladder is configured as a head-wearable element having one or more flexible flaps configured to extend over a forehead or a scalp of the organism.

Embodiment 10. The wearable heating or cooling device of embodiment 1, wherein the body made of the polymer defines an internal cavity that contains the first metal heat exchanger, the Peltier element, and at least a portion of the second metal heat exchanger as a sealed module that is fluidically isolated from ambient air.

Embodiment 11. The wearable heating or cooling device of embodiment 1, wherein the device is a joint brace configured to be worn on a joint of the organism, and wherein the bladder is a part of the joint brace, the brace including a pneumatic actuator configured to maintain the bladder in contact with the joint.

Embodiment 12. The wearable heating or cooling device of embodiment 1, wherein the second metal heat exchanger comprises a gyroid lattice defining air passages, the gyroid lattice having a thermally conductive coating.

Embodiment 13. The wearable heating or cooling device of embodiment 12, wherein the thermally conductive coating comprises at least one of nickel, copper, aluminum, or graphene.

Embodiment 14. The wearable heating or cooling device of embodiment 1, wherein the air-to-liquid heat exchanger comprises the fan or blower associated with a first heat sink and another fan or blower associated with a second heat sink, the first heat sink and the second heat sink being thermally coupled to the second side of the Peltier element.

Embodiment 15. The wearable heating or cooling device of embodiment 14, wherein the first and second heat sinks are coupled to opposing ends of a plurality of heat pipes with the Peltier device coupled to the heat pipes between the first and second heat sinks, and wherein opposing ends of the plurality of heat pipes are bent toward the body.

Embodiment 16. The wearable heating or cooling device of embodiment 1, wherein the polymer body comprises a polymer overmold that at least partially encapsulates the first metal heat exchanger and defines one or more fluid manifolds that couple the first metal heat exchanger to the working liquid flow path.

Embodiment 17. The wearable heating or cooling device of embodiment 1, further comprising a quick-disconnect fluid coupling between the bladder and the air-to-liquid heat exchanger, the quick-disconnect fluid coupling including an automatic shutoff valve configured to prevent leakage when disconnected.

Embodiment 18. The wearable heating or cooling device of embodiment 1, further comprising a heat pipe thermally coupled to the second metal heat exchanger, the heat pipe configured to conduct heat to an auxiliary heat sink positioned remotely from the air-to-liquid heat exchanger.

Embodiment 19. The wearable heating or cooling device of embodiment 18, further comprising a crush plate configured to deform a wall of the heat pipe to increase a thermal contact area between the heat pipe and the auxiliary heat sink.

Embodiment 20. The wearable heating or cooling device of embodiment 1, wherein the bladder comprises a plurality of discrete bladder segments fluidically coupled in parallel to the air-to-liquid heat exchanger to provide redundant flow paths.

Embodiment 21. The wearable heating or cooling device of embodiment 1, further comprising a wearable mounting structure that positions the air-to-liquid heat exchanger remotely from the bladder on a torso or waist of the organism, with one or more flexible tubes fluidically coupling the air-to-liquid heat exchanger to the bladder.

What is claimed is:

1. A wearable heating or cooling device, comprising:
a bladder configured to be positioned adjacent an organism, the bladder defining part of a working liquid flow path; and
an air-to-liquid heat exchanger fluidically coupled to the bladder, the air-to-liquid heat exchanger comprising:
a pump configured to circulate a working liquid through the working liquid flow path;
a body made of a polymer;
a first metal heat exchanger having a plurality of protrusions positioned in a portion of the working liquid flow path inside the body;
a Peltier device having a first side adjacent the first metal heat exchanger and a second side opposite the first side;
a fan or blower coupled to the air-to-liquid heat exchanger and configured to force air along an air flow path; and
a second metal heat exchanger adjacent the second side of the Peltier device, the second metal heat exchanger having a plurality of protrusions positioned in the air flow path of the fan or blower.

2. The wearable heating or cooling device of claim 1, wherein the bladder comprises a pair of polymer sheets sealed to one another to define a serpentine liquid channel forming at least a portion of the working liquid flow path.

3. The wearable heating or cooling device of claim 1, wherein the bladder comprises a thermoplastic polyurethane film and a textile-backed outer layer configured to be positioned against the organism.

4. The wearable heating or cooling device of claim 1, wherein the bladder comprises a multi-layer laminate including an inner barrier layer configured to reduce permeation of the working liquid and an outer layer configured to be heat-sealable.

5. The wearable heating or cooling device of claim 1, wherein the bladder includes one or more internal inserts positioned within the bladder to define at least one of (i) a flow-distribution region, or (ii) a baffle region that inhibits ballooning of the bladder.

6. The wearable heating or cooling device of claim 1, wherein the first metal heat exchanger comprises aluminum and the plurality of protrusions comprises pins or posts integrally formed with the first metal heat exchanger.

7. The wearable heating or cooling device of claim 1, wherein the second metal heat exchanger comprises copper and the plurality of protrusions comprises fins configured to promote turbulent airflow in the air flow path.

8. The wearable heating or cooling device of claim 1, wherein the bladder is configured as a neck-wearable band having a curved profile that wraps at least partially around a neck of the organism.

9. The wearable heating or cooling device of claim 1, wherein the bladder is configured as a head-wearable element having one or more flexible flaps configured to extend over a forehead or a scalp of the organism.

10. The wearable heating or cooling device of claim 1, wherein the body made of the polymer defines an internal cavity that contains the first metal heat exchanger, the Peltier device, and at least a portion of the second metal heat exchanger as a sealed module that is fluidically isolated from ambient air.

11. The wearable heating or cooling device of claim 1, wherein the device is a joint brace configured to be worn on a joint of the organism, and wherein the bladder is a part of the joint brace, the brace including a pneumatic actuator configured to maintain the bladder in contact with the joint.

12. The wearable heating or cooling device of claim 1, wherein the second metal heat exchanger comprises a gyroid lattice defining air passages, the gyroid lattice having a thermally conductive coating.

13. The wearable heating or cooling device of claim 12, wherein the thermally conductive coating comprises at least one of nickel, copper, aluminum, or graphene.

14. The wearable heating or cooling device of claim 1, wherein the air-to-liquid heat exchanger comprises the fan or blower associated with a first heat sink and another fan or blower associated with a second heat sink, the first heat sink and the second heat sink being thermally coupled to the second side of the Peltier device.

15. The wearable heating or cooling device of claim 14, wherein the first and second heat sinks are coupled to opposing ends of a plurality of heat pipes with the Peltier device coupled to the heat pipes between the first and second heat sinks, and wherein opposing ends of the plurality of heat pipes are bent toward the body.

16. The wearable heating or cooling device of claim 1, wherein the polymer body comprises a polymer overmold that at least partially encapsulates the first metal heat exchanger and defines one or more fluid manifolds that couple the first metal heat exchanger to the working liquid flow path.

17. The wearable heating or cooling device of claim 1, further comprising a quick-disconnect fluid coupling between the bladder and the air-to-liquid heat exchanger, the quick-disconnect fluid coupling including an automatic shutoff valve configured to prevent leakage when disconnected.

18. The wearable heating or cooling device of claim 1, further comprising a heat pipe thermally coupled to the second metal heat exchanger, the heat pipe configured to conduct heat to an auxiliary heat sink positioned remotely from the air-to-liquid heat exchanger.

19. The wearable heating or cooling device of claim 18, further comprising a crush plate configured to deform a wall of the heat pipe to increase a thermal contact area between the heat pipe and the auxiliary heat sink.

20. The wearable heating or cooling device of claim 1, wherein the bladder comprises a plurality of discrete bladder segments fluidically coupled in parallel to the air-to-liquid heat exchanger to provide redundant flow paths.

21. The wearable heating or cooling device of claim 1, further comprising a wearable mounting structure that positions the air-to-liquid heat exchanger remotely from the bladder on a torso or waist of the organism, with one or more flexible tubes fluidically coupling the air-to-liquid heat exchanger to the bladder.
A
